SEQUENCE LISTING (122 sequences)

SEQ ID NO: 1 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 2 - protein, 950 aa, Homo sapiens
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 3 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 4 - protein, 398 aa, Homo sapiens
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITTGSENLHVSHILQKAKIEVSEDGTKASAATTAILIARSSPPWFIVDRPFLFFIRHNPTGAVLFMGQINKP

SEQ ID NO: 5 - protein, 4684 aa, Homo sapiens
MVAGMLMPRDQLRAIYEVLFREGVMVAKKDRRPRSLHPHVPGVTNLQVMRAMASLRARGLVRETFAWCHFYWYLTNEGIAHLRQYLHLPPEIVPASLQRVRRPVAMVMPARRTPHVQAVQGPLGSPPKRGPLPTEEQRVYRRKELEEVSPETPVVPATTQRTLARPGPEPAPATDERDRVQKKTFTKWVNKHLIKAQRHISDLYEDLRDGHNLISLLEVLSGDSLPREKGRMRFHKLQNVQIALDYLRHRQVKLVNIRNDDIADGNPKLTLGLIWTIILHFQISDIQVSGQSEDMTAKEKLLLWSQRMVEGYQGLRCDNFTSSWRDGRLFNAIIHRHKPLLIDMNKVYRQTNLENLDQAFSVAERDLGVTRLLDPEDVDVPQPDEKSIITYVSSLYDAMPRVPDVQDGVRANELQLRWQEYRELVLLLLQWMRHHTAAFEERRFPSSFEEIEILWSQFLKFKEMELPAKEADKNRSKGIYQSLEGAVQAGQLKVPPGYHPLDVEKEWGKLHVAILEREKQLRSEFERLECLQRIVTKLQMEAGLCEEQLNQADALLQSDVRLLAAGKVPQRAGEVERDLDKADSMIRLLFNDVQTLKDGRHPQGEQMYRRVYRLHERLVAIRTEYNLRLKAGVAAPATQVAQVTLQSVQRRPELEDSTLRYLQDLLAWVEENQHRVDGAEWGVDLPSVEAQLGSHRGLHQSIEEFRAKIERARSDEGQLSPATRGAYRDCLGRLDLQYAKLLNSSKARLRSLESLHSFVAAATKELMWLNEKEEEEVGFDWSDRNTNMTAKKESYSALMRELELKEKKIKELQNAGDRLLREDHPARPTVESFQAALQTQWSWMLQLCCCIEAHLKENAAYFQFFSDVREAEGQLQKLQEALRRKYSCDRSATVTRLEDLLQDAQDEKEQLNEYKGHLSGLAKRAKAVVQLKPRHPAHPMRGRLPLLAVCDYKQVEVTVHKGDECQLVGPAQPSHWKVLSSSGSEAAVPSVCFLVPPPNQEAQEAVTRLEAQHQALVTLWHQLHVDMKSLLAWQSLRRDVQLIRSWSLATFRTLKPEEQRQALHSLELHYQAFLRDSQDAGGFGPEDRLMAEREYGSCSHHYQQLLQSLEQGAQEESRCQRCISELKDIRLQLEACETRTVHRLRLPLDKEPARECAQRIAEQQKAQAEVEGLGKGVARLSAEAEKVLALPEPSPAAPTLRSELELTLGKLEQVRSLSAIYLEKLKTISLVIRGTQGAEEVLRAHEEQLKEAQAVPATLPELEATKASLKKLRAQAEAQQPTFDALRDELRGAQEVGERLQQRHGERDVEVERWRERVAQLLERWQAVLAQTDVRQRELEQLGRQLRYYRESADPLGAWLQDARRRQEQIQAMPLADSQAVREQLRQEQALLEEIERHGEKVEECQRFAKQYINAIKDYELQLVTYKAQLEPVASPAKKPKVQSGSESVIQEYVDLRTHYSELTTLTSQYIKFISETLRRMEEEERLAEQQRAEERERLAEVEAALEKQRQLAEAHAQAKAQAEREAKELQQRMQEEVVRREEAAVDAQQQKRSIQEELQQLRQSSEAEIQAKARQAEAAERSRLRIEEEIRVVRLQLEATERQRGGAEGELQALRARAEEAEAQKRQAQEEAERLRRQVQDESQRKRQAEVELASRVKAEAEAAREKQRALQALEELRLQAEEAERRLRQAEVERARQVQVALETAQRSAEAELQSKRASFAEKTAQLERSLQEEHVAVAQLREEAERRAQQQAEAERAREEAERELERWQLKANEALRLRLQAEEVAQQKSLAQAEAEKQKEEAEREARRRGKAEEQAVRQRELAEQELEKQRQLAEGTAQQRLAAEQELIRLRAETEQGEQQRQLLEEELARLQREAAAATQKRQELEAELAKVRAEMEVLLASKARAEEESRSTSEKSKQRLEAEAGRFRELAEEAARLRALAEEAKRQRQLAEEDAARQRAEAERVLAEKLAAIGEATRLKTEAEIALKEKEAENERLRRLAEDEAFQRRRLEEQAAQHKADIEERLAQLRKASDSELERQKGLVEDTLRQRRQVEEEILALKASFEKAAAGKAELELELGRIRSNAEDTLRSKEQAELEAARQRQLAAEEERRRREAEERVQKSLAAEEEAARQRKAALEEVERLKAKVEEARRLRERAEQESARQLQLAQEAAQKRLQAEEKAHAFAVQQKEQELQQTLQQEQSVLDQLRGEAEAARRAAEEAEEARVQAEREAAQSRRQVEEAERLKQSAEEQAQARAQAQAAAEKLRKEAEQEAARRAQAEQAALRQKQAADAEMEKHKKFAEQTLRQKAQVEQELTTLRLQLEETDHQKNLLDEELQRLKAEATEAARQRSQVEEELFSVRVQMEELSKLKARIEAENRALILRDKDNTQRFLQEEAEKMKQVAEEAARLSVAAQEAARLRQLAEEDLAQQRALAEKMLKEKMQAVQEATRLKAEAELLQQQKELAQEQARRLQEDKEQMAQQLAEETQGFQRTLEAERQRQLEMSAEAERLKLRVAEMSRAQARAEEDAQRFRKQAEEIGEKLHRTELATQEKVTLVQTLEIQRQQSDHDAERLREAIAELEREKEKLQQEAKLLQLKSEEMQTVQQEQLLQETQALQQSFLSEKDSLLQRERFIEQEKAKLEQLFQDEVAKAQQLREEQQRQQQQMEQERQRLVASMEEARRRQHEAEEGVRRKQEELQQLEQQRRQQEELLAEENQRLREQLQLLEEQHRAALAHSEEVTASQVAATKTLPNGRDALDGPAAEAEPEHSFDGLRRKVSAQRLQEAGILSAEELQRLAQGHTTVDELARREDVRHYLQGRSSIAGLLLKATNEKLSVYAALQRQLLSPGTALILLEAQAASGFLLDPVRNRRLTVNEAVKEGVVGPELHHKLLSAERAVTGYKDPYTGQQISLFQAMQKGLIVREHGIRLLEAQIATGGVIDPVHSHRVPVDVAYRRGYFDEEMNRVLADPSDDTKGFFDPNTHENLTYLQLLERCVEDPETGLCLLPLTDKAAKGGELVYTDSEARDVFEKATVSAPFGKFQGKTVTIWEIINSEYFTAEQRRDLLRQFRTGRITVEKIIKIIITVVEEQEQKGRLCFEGLRSLVPAAELLESRVIDRELYQQLQRGERSVRDVAEVDTVRRALRGANVIAGVWLEEAGQKLSIYNALKKDLLPSDMAVALLEAQAGTGHIIDPATSARLTVDEAVRAGLVGPEFHEKLLSAEKAVTGYRDPYTGQSVSLFQALKKGLIPREQGLRLLDAQLSTGGIVDPSKSHRVPLDVACARGCLDEETSRALSAPRADAKAYSDPSTGEPATYGELQQRCRPDQLTGLSLLPLSEKAARARQEELYSELQARETFEKTPVEVPVGGFKGRTVTVWELISSEYFTAEQRQELLRQFRTGKVTVEKVIKILITIVEEVETLRQERLSFSGLRAPVPASELLASGVLSRAQFEQLKDGKTTVKDLSELGSVRTLLQGSGCLAGIYLEDTKEKVSIYEAMRRGLLRATTAALLLEAQAATGFLVDPVRNQRLYVHEAVKAGVVGPELHEQLLSAEKAVTGYRDPYSGSTISLFQAMQKGLVLRQHGIRLLEAQIATGGIIDPVHSHRVPVDVAYQRGYFSEEMNRVLADPSDDTKGFFDPNTHENLTYRQLLERCVEDPETGLRLLPLKGAEKAEVVETTQVYTEEETRRAFEETQIDIPGGGSHGGSTMSLWEVMQSDLIPEEQRAQLMADFQAGRVTKERMIIIIIEIIEKTEIIRQQGLASYDYVRRRLTAEDLFEARIISLETYNLLREGTRSLREALEAESAWCYLYGTGSVAGVYLPGSRQTLSIYQALKKGLLSAEVARLLLEAQAATGFLLDPVKGERLTVDEAVRKGLVGPELHDRLLSAERAVTGYRDPYTEQTISLFQAMKKELIPTEEALRLLDAQLATGGIVDPRLGFHLPLEVAYQRGYLNKDTHDQLSEPSEVRSYVDPSTDERLSYTQLLRRCRRDDGTGQLLLPLSDARKLTFRGLRKQITMEELVRSQVMDEATALQLREGLTSIEEVTKNLQKFLEGTSCIAGVFVDATKERLSVYQAMKKGIIRPGTAFELLEAQAATGYVIDPIKGLKLTVEEAVRMGIVGPEFKDKLLSAERAVTGYKDPYSGKLISLFQAMKKGLILKDHGIRLLEAQIATGGIIDPEESHRLPVEVAYKRGLFDEEMNEILTDPSDDTKGFFDPNTEENLTYLQLMERCITDPQTGLCLLPLKEKKRERKTSSKSSVRKRRVVIVDPETGKEMSVYEAYRKGLIDHQTYLELSEQECEWEEITISSSDGVVKSMIIDRRSGRQYDIDDAIAKNLIDRSALDQYRAGTLSITEFADMLSGNAGGFRSRSSSVGSSSSYPISPAVSRTQLASWSDPTEETGPVAGILDTETLEKVSITEAMHRNLVDNITGQRLLEAQACTGGIIDPSTGERFPVTDAVNKGLVDKIMVDRINLAQKAFCGFEDPRTKTKMSAAQALKKGWLYYEAGQRFLEVQYLTGGLIEPDTPGRVPLDEALQRGTVDARTAQKLRDVGAYSKYLTCPKTKLKISYKDALDRSMVEEGTGLRLLEAAAQSTKGYYSPYSVSGSGSTAGSRTGSRTGSRAGSRRGSFDATGSGFSMTFSSSSYSSSGYGRRYASGSSASLGGPESAVA

SEQ ID NO: 6 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 7 - protein, 710 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 8 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEDELYAQKLKYKAISEELDHALNDMTSI

SEQ ID NO: 9 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 10 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 11 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 12 - protein, 323 aa, Homo sapiens
MQAAWLLGALVVPQLLGFGHGARGAEREWEGGWGGAQEEEREREALMLKHLQEALGLPAGRGDENPAGTVEGKEDWEMEEDQGEEEEEEATPTPSSGPSPSPTPEDIVTYILGRLAGLDAGLHQLHVRLHALDTRVVELTQGLRQLRNAAGDTRDAVQALQEAQGRAEREHGRLEGCLKGLRLGHKCFLLSRDFEAQAAAQARCTARGGSLAQPADRQQMEALTRYLRAALAPYNWPVWLGVHDRRAEGLYLFENGQRVSFFAWHRSPRPELGAQPSASPHPLSPDQPNGGTLENCVAQASDDGSWWDHDCQRRLYYVCEFPF

SEQ ID NO: 13 - protein, 2468 aa, Homo sapiens
MATVVVEATEPEPSGSIANPAASTSPSLSHRFLDSKFYLLVVVGEIVTEEHLRRAIGNIELGIRSWDTNLIECNLDQELKLFVSRHSARFSPEVPGQKILHHRSDVLETVVLINPSDEAVSTEVRLMITDAARHKLLVLTGQCFENTGELILQSGSFSFQNFIEIFTDQEIGELLSTTHPANKASLTLFCPEEGDWKNSNLDRHNLQDFINIKLNSASILPEMEGLSEFTEYLSESVEVPSPFDILEPPTSGGFLKLSKPCCYIFPGGRGDSALFAVNGFNMLINGGSERKSCFWKLIRHLDRVDSILLTHIGDDNLPGINSMLQRKIAELEEEQSQGSTTNSDWMKNLISPDLGVVFLNVPENLKNPEPNIKMKRSIEEACFTLQYLNKLSMKPEPLFRSVGNTIDPVILFQKMGVGKLEMYVLNPVKSSKEMQYFMQQWTGTNKDKAEFILPNGQEVDLPISYLTSVSSLIVWHPANPAEKIIRVLFPGNSTQYNILEGLEKLKHLDFLKQPLATQKDLTGQVPTPVVKQTKLKQRADSRESLKPAAKPLPSKSVRKESKEETPEVTKVNHVEKPPKVESKEKVMVKKDKPIKTETKPSVTEKEVPSKEEPSPVKAEVAEKQATDVKPKAAKEKTVKKETKVKPEDKKEEKEKPKKEVAKKEDKTPIKKEEKPKKEEVKKEVKKEIKKEEKKEPKKEVKKETPPKEVKKEVKKEEKKEVKKEEKEPKKEIKKLPKDAKKSSTPLSEAKKPAALKPKVPKKEESVKKDSVAAGKPKEKGKIKVIKKEGKAAEAVAAAVGTGATTAAVMAAAGIAAIGPAKELEAERSLMSSPEDLTKDFEELKAEEVDVTKDIKPQLELIEDEEKLKETEPVEAYVIQKEREVTKGPAESPDEGITTTEGEGECEQTPEELEPVEKQGVDDIEKFEDEGAGFEESSETGDYEEKAETEEAEEPEEDGEEHVCVSASKHSPTEDEESAKAEADAYIREKRESVASGDDRAEEDMDEAIEKGEAEQSEEEADEEDKAEDAREEEYEPEKMEAEDYVMAVVDKAAEAGGAEEQYGFLTTPTKQLGAQSPGREPASSIHDETLPGGSESEATASDEENREDQPEEFTATSGYTQSTIEISSEPTPMDEMSTPRDVMSDETNNEETESPSQEFVNITKYESSLYSQEYSKPADVTPLNGFSEGSKTDATDGKDYNASASTISPPSSMEEDKFSRSALRDAYCSEVKASTTLDIKDSISAVSSEKVSPSKSPSLSPSPPSPLEKTPLGERSVNFSLTPNEIKVSAEAEVAPVSPEVTQEVVEEHCASPEDKTLEVVSPSQSVTGSAGHTPYYQSPTDEKSSHLPTEVIEKPPAVPVSFEFSDAKDENERASVSPMDEPVPDSESPIEKVLSPLRSPPLIGSESAYESFLSADDKASGRGAESPFEEKSGKQGSPDQVSPVSEMTSTSLYQDKQEGKSTDFAPIKEDFGQEKKTDDVEAMSSQPALALDERKLGDVSPTQIDVSQFGSFKEDTKMSISEGTVSDKSATPVDEGVAEDTYSHMEGVASVSTASVATSSFPEPTTDDVSPSLHAEVGSPHSTEVDDSLSVSVVQTPTTFQETEMSPSKEECPRPMSISPPDFSPKTAKSRTPVQDHRSEQSSMSIEFGQESPEQSLAMDFSRQSPDHPTVGAGVLHITENGPTEVDYSPSDMQDSSLSHKIPPMEEPSYTQDNDLSELISVSQVEASPSTSSAHTPSQIASPLQEDTLSDVAPPRDMSLYASLTSEKVQSLEGEKLSPKSDISPLTPRESSPLYSPTFSDSTSAVKEKTATCHSSSSPPIDAASAEPYGFRASVLFDTMQHHLALNRDLSTPGLEKDSGGKTPGDFSYAYQKPEETTRSPDEEDYDYESYEKTTRTSDVGGYYYEKIERTTKSPSDSGYSYETIGKTTKTPEDGDYSYEIIEKTTRTPEEGGYSYDISEKTTSPPEVSGYSYEKTERSRRLLDDISNGYDDSEDGGHTLGDPSYSYETTEKITSFPESEGYSYETSTKTTRTPDTSTYCYETAEKITRTPQASTYSYETSDLCYTAEKKSPSEARQDVDLCLVSSCEYKHPKTELSPSFINPNPLEWFASEEPTEESEKPLTQSGGAPPPPGGKQQGRQCDETPPTSVSESAPSQTDSDVPPETEECPSITADANIDSEDESETIPTDKTVTYKHMDPPPAPVQDRSPSPRHPDVSMVDPEALAIEQNLGKALKKDLKEKTKTKKPGTKTKSSSPVKKSDGKSKPLAASPKPAGLKESSDKVSRVASPKKKESVEKAAKPTTTPEVKAARGEEKDKETKNAANASASKSAKTATAGPGTTKTTKSSAVPPGLPVYLDLCYIPNHSNSKNVDVEFFKRVRSSYYVVSGNDPAAEEPSRAVLDALLEGKAQWGSNMQVTLIPTHDSEVMREWYQETHEKQQDLNIMVLASSSTVVMQDESFPACKIEL

SEQ ID NO: 14 - protein, 215 aa, Homo sapiens
MPGEATETVPATEQELPQPQAETGSGTESDSDESVPELEEQDSTQATTQQAQLAAAAEIDEEPVSKAKQSRSEKKARKAMSKLGLRQVTGVTRVTIRKSKNILFVITKPDVYKSPASDTYIVFGEAKIEDLSQQAQLAAAEKFKVQGEAVSNIQENTQTPTVQEESEEEEVDETGVEVKDIELVMSQANVSRAKAVRALKNNSNDIVNAIMELTM

SEQ ID NO: 15 - protein, 234 aa, Homo sapiens
MAERGYSFSLTTFSPSGKLVQIEYALAAVAGGAPSVGIKAANGVVLATEKKQKSILYDERSVHKVEPITKHIGLVYSGMGPDYRVLVHRARKLAQQYYLVYQEPIPTAQLVQRVASVMQEYTQSGGVRPFGVSLLICGWNEGRPYLFQSDPSGAYFAWKATAMGKNYVNGKTFLEKRYNEDLELEDAIHTAILTLKESFEGQMTEDNIEVGICNEAGFRRLTPTEVKDYLAAIA

SEQ ID NO: 16 - protein, 297 aa, Homo sapiens [sequence omitted]

SEQ ID NO: 17 - protein, 151 aa, Homo sapiens
MCDFTEDQTAEFKEAFQLFDRTGDGKILYSQCGDVMRALGQNPTNAEVLKVLGNPKSDEMNVKVLDFEHFLPMLQTVAKNKDQGTYEDYVEGLRVFDKEGNGTVMGAEIRHVLVTLGEKMTEEEVEMLVAGHEDSNGCINYEAFVRHILSG

SEQ ID NO: 18 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 19 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 20 - protein, 390 aa, Homo sapiens
MEDPTLYIVERPLPGYPDAEAPEPSSAGAQAAEEPSGAGSEELIKSDQVNGVLVLSLLDKIIGAVDQIQLTQAQLEERQAEMEGAVQSIQGELSKLGKAHATTSNTVSKLLEKVRKVSVNVKTVRGSLERQAGQIKKLEVNEAELLRRRNFKVMIYQDEVKLPAKLSISKSLKESEALPEKEGEELGEGERPEEDAAALELSSDEAVEVEEVIEESRAERIKRSGLRRVDDFKKAFSKEKMEKTKVRTRENLEKTRLKTKENLEKTRHTLEKRMNKLGTRLVPAERREKLKTSRDKLRKSFTPDHVVYARSKTAVYKVPPFTFHVKKIREGQVEVLKATEMVEVGADDDEGGAERGEAGDLRRGSSPDVHALLEITEESDAVLVDKSDSD

SEQ ID NO: 21 - protein, 461 aa, Homo sapiens
MPPSGPRGTLLLLPLLLLLLLRAVLAVPLERGAPNKEETPATESPDTGLYYHRYLQEVIDVLETDGHFREKLQAANAEDIKSGKLSRELDFVSHHVRTKLDELKRQEVSRLRMLLKAKMDAEQDPNVQVDHLNLLKQFEHLDPQNQHTFEARDLELLIQTATRDLAQYDAAHHEEFKRYEMLKEHERRRYLESLGEEQRKEAERKLEEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGDTGEGWETVEMHPAYTEEELRRFEEELAAREAELNAKAQRLSQETEALGRSQGRLEAQKRELQQAVLHMEQRKQQQQQQQGHKAPAAHPEGQLKFHPDTDDVPVPAPAGDQKEVDTSEKKLLERLPEVEVPQHL

SEQ ID NO: 22 - protein, 645 aa, Homo sapiens
MRPRKAFLLLLLLGLVQLLAVAGAEGPDEDSSNRENAIEDEEEEEEEDDDEEEDDLEVKEENGVLVLNDANFDNFVADKDTVLLEFYAPWCGHCKQFAPEYEKIANILKDKDPPIPVAKIDATSASVLASRFDVSGYPTIKILKKGQAVDYEGSRTQEEIVAKVREVSQPDWTPPPEVTLVLTKENFDEVVNDADIILVEFYAPWCGHCKKLAPEYEKAAKELSKRSPPIPLAKVDATAETDLAKRFDVSGYPTLKIFRKGRPYDYNGPREKYGIVDYMIEQSGPPSKEILTLKQVQEFLKDGDDVIIIGVFKGESDPAYQQYQDAANNLREDYKFHHTFSTEIAKFLKVSQGQLVVMQPEKFQSKYEPRSHMMDVQGSTQDSAIKDFVLKYALPLVGHRKVSNDAKRYTRRPLVVVYYSVDFSFDYRAATQFWRSKVLEVAKDFPEYTFAIADEEDYAGEVKDLGLSESGEDVNAAILDESGKKFAMEPEEFDSDTLREFVTAFKKGKLKPVIKSQPVPKNNKGPVKVVVGKTFDSIVMDPKKDVLIEFYAPWCGHCKQLEPVYNSLAKKYKGQKGLVIAKMDATANDVPSDRYKVEGFPTIYFAPSGDKKNPVKFEGGDRDLEHLSKFIEEHATKLSRTKEEL

SEQ ID NO: 23 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 24 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

SEQ ID NO: 25 - protein, 281 aa, Homo sapiens
MATNFLAHEKIWFDKFKYDDAERRFYEQMNGPVAGASRQENGASVILRDIARARENIQKSLAGSSGPGASSGTSGDHGELVVRIASLEVENQSLRGVVQELQQAISKLEARLNVLEKSSPGHRATAPQTQHVSPMRQVEPPAKKPATPAEDDEDDDIDLFGSDNEEEDKEAAQLREERLRQYAEKKAKKPALVAKSSILLDVKPWDDETDMAQLEACVRSIQLDGLVWGASKLVPVGYGIRKLQIQCVVEDDKVGTDLLEEEITKFEEHVQSVDIAAFNKI

SEQ ID NO: 26 - protein, 286 aa, Homo sapiens
MAEDGEEAEFHFAALYISGQWPRLRADTDLQRLGSSAMAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 27 - protein, 148 aa, Homo sapiens
MFRRPVLQVLRQFVRHESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 28 - protein, 276 aa, Homo sapiens
MALLDVCGAPRGQRPESALPVAGSGRRSDPGHYSFSMRSPELALPRGMQPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 29 - protein, 524 aa, Homo sapiens
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 30 - protein, 277 aa, Homo sapiens
MAAVSVYAPPVGGFSFDNCRRNAVLEADFAKRGYKLPKVRKTGTTIAGVVYKDGIVLGADTRATEGMVVADKNCSKIHFISPNIYCCGAGTAADTDMTTQLISSNLELHSLSTGRLPRVVTANRMLKQMLFRYQGYIGAALVLGGVDVTGPHLYSIYPHGSTDKLPYVTMGSGSLAAMAVFEDKFRPDMEEEEAKNLVSEAIAAGIFNDLGSGSNIDLCVISKNKLDFLRPYTVPNKKGTRLGRYRCEKGTTAVLTEKITPLEIEVLEETVQTMDTS

SEQ ID NO: 31 - protein, 286 aa, Homo sapiens
MADFDDRVSDEEKVRIAAKFITHAPPGEFNEVFNDVRLLLNNDNLLREGAAHAFAQYNMDQFTPVKIEGYEDQVLITEHGDLGNSRFLDPRNKISFKFDHLRKEASDPQPEEADGGLKSWRESCDSALRAYVKDHYSNGFCTVYAKTIDGQQTIIACIESHQFQPKNFWNGRWRSEWKFTITPPTAQVVGVLKIQVHYYEDGNVQLVSHKDVQDSLTVSNEAQTAKEFIKIIENAENEYQTAISENYQTMSDTTFKALRRQLPVTRTKIDWNKILSYKIGKEMQNA

SEQ ID NO: 32 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 33 - protein, 613 aa, Homo sapiens
MAQSRVLLLLLLLPPQLHLGPVLAVRAPGFGRSGGHSLSPEENEFAEEEPVLVLSPEEPGPGPAAVSCPRDCACSQEGVVDCGGIDLREFPGDLPEHTNHLSLQNNQLEKIYPEELSRLHRLETLNLQNNRLTSRGLPEKAFEHLTNLNYLYLANNKLTLAPRFLPNALISVDFAANYLTKIYGLTFGQKPNLRSVYLHNNKLADAGLPDNMFNGSSNVEVLILSSNFLRHVPKHLPPALYKLHLKNNKLEKIPPGAFSELSSLRELYLQNNYLTDEGLDNETFWKLSSLEYLDLSSNNLSRVPAGLPRSLVLLHLEKNAIRSVDANVLTPIRSLEYLLLHSNQLREQGIHPLAFQGLKRLHTVHLYNNALERVPSGLPRRVRTLMILHNQITGIGREDFATTYFLEELNLSYNRITSPQVHRDAFRKLRLLRSLDLSGNRLHTLPPGLPRNVHVLKVKRNELAALARGALVGMAQLRELYLTSNRLRSRALGPRAWVDLAHLQLLDIAGNQLTEIPEGLPESLEYLYLQNNKISAVPANAFDSTPNLKGIFLRFNKLAVGSVVDSAFRRLKHLQVLDIEGNLEFGDISKDRGRLGKEKEEEEEEEEEEEETR

SEQ ID NO: 34 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 35 - protein, 420 aa, Homo sapiens
MRWRTILLQYCFLLITCLLTALEAVPIDIDKTKVQNIHPVESAKIEPPDTGLYYDEYLKQVIDVLETDKHFREKLQKADIEEIKSGRLSKELDLVSHHVRTKLDELKRQEVGRLRMLIKAKLDSLQDIGMDHQALLKQFDHLNHLNPDKFESTDLDMLIKAATSDLEHYDKTRHEEFKKYEMMKEHERREYLKTLNEEKRKEEESKFEEMKKKHENHPKVNHPGSKDQLKEVWEETDGLDPNDFDPKTFFKLHDVNSDGFLDEQELEALFTKELEKVYDPKNEEDDMVEMEEERLRMREHVMSEVDTNKDRLVTLEEFLKATEKKEFLEPDSWETLDQQQFFTEEELKEYENIIALQENELKKKADELQKQKEELQRQHDQLEAQKLEYHQVIQQMEQKKLQQGIPPSGPAGELKFEPHI

SEQ ID NO: 36 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 37 - protein, 5890 aa, Homo sapiens
MEKEETTRELLLPNWQGSGSHGLTIAQRDDGVFVQEVTQNSPAARTGVVKEGDQIVGATIYFDNLQSGEVTQLLNTMGHHTVGLKLHRKGDRSPEPGQTWTREVFSSCSSEVVLSGDDEEYQRIYTTKIKPRLKSEDGVEGDLGETQSRTITVTRRVTAYTVDVTGREGAKDIDISSPEFKIKIPRHELTEISNVDVETQSGKTVIRLPSGSGAASPTGSAVDIRAGAISASGPELQGAGHSKLQVTMPGIKVGGSGVNVNAKGLDLGGRGGVQVPAVDISSSLGGRAVEVQGPSLESGDHGKIKFPTMKVPKFGVSTGREGQTPKAGLRVSAPEVSVGHKGGKPGLTIQAPQLEVSVPSANIEGLEGKLKGPQITGPSLEGDLGLKGAKPQGHIGVDASAPQIGGSITGPSVEVQAPDIDVQGPGSKLNVPKMKVPKFSVSGAKGEETGIDVTLPTGEVTVPGVSGDVSLPEIATGGLEGKMKGTKVKTPEMIIQKPKISMQDVDLSLGSPKLKGDIKVSAPGVQGDVKGPQVALKGSRVDIETPNLEGTLTGPRLGSPSGKTGTCRISMSEVDLNVAAPKVKGGVDVTLPRVEGKVKVPEVDVRGPKVDVSAPDVEAHGPEWNLKMPKMKMPTFSTPGAKGEGPDVHMTLPKGDISISGPKVNVEAPDVNLEGLGGKLKGPDVKLPDMSVKTPKISMPDVDLHVKGTKVKGEYDVTVPKLEGELKGPKVDIDAPDVDVHGPDWHLKMPKMKMPKFSVPGFKAEGPEVDVNLPKADVDISGPKIDVTAPDVSIEEPEGKLKGPKFKMPEMNIKVPKISMPDVDLHLKGPNVKGEYDVTMPKVESEIKVPDVELKSAKMDIDVPDVEVQGPDWHLKMPKMKMPKFSMPGFKAEGPEVDVNLPKADVDISGPKVGVEVPDVNIEGPEGKLKGPKFKMPEMNIKAPKISMPDVDLHMKGPKVKGEYDMTVPKLEGDLKGPKVDVSAPDVEMQGPDWNLKMPKIKMPKFSMPSLKGEGPEFDVNLSKANVDISAPKVDTNAPDLSLEGPEGKLKGPKFKMPEMHFRAPKMSLPDVDLDLKGPKMKGNVDISAPKIEGEMQVPDVDIRGPKVDIKAPDVEGQGLDWSLKIPKMKMPKFSMPSLKGEGPEVDVNLPKADVVVSGPKVDIEAPDVSLEGPEGKLKGPKFKMPEMHFKTPKISMPDVDLHLKGPKVKGDVDVSVPKVEGEMKVPDVEIKGPKMDIDAPDVEVQGPDWHLKMPKMKMPKFSMPGFKGEGREVDVNLPKADIDVSGPKVDVEVPDVSLEGPEGKLKGPKFKMPEMHFKAPKISMPDVDLNLKGPKLKGDVDVSLPEVEGEMKVPDVDIKGPKVDISAPDVDVHGPDWHLKMPKVKMPKFSMPGFKGEGPEVDVKLPKADVDVSGPKMDAEVPDVNIEGPDAKLKGPKFKMPEMSIKPQKISIPDVGLHLKGPKMKGDYDVTVPKVEGEIKAPDVDIKGPKVDINAPDVEVHGPDWHLKMPKVKMPKFSMPGFKGEGPEVDMNLPKADLGVSGPKVDIDVPDVNLEAPEGKLKGPKFKMPSMNIQTHKISMPDVGLNLKAPKLKTDVDVSLPKVEGDLKGPEIDVKAPKMDVNVGDIDIEGPEGKLKGPKFKMPEMHFKAPKISMPDVDLHLKGPKVKGDMDVSVPKVEGEMKVPDVDIKGPKVDIDAPDVEVHDPDWHLKMPKMKMPKFSMPGFKAEGPEVDVNLPKADIDVSGPSVDTDAPDLDIEGPEGKLKGSKFKMPKLNIKAPKVSMPDVDLNLKGPKLKGEIDASVPELEGDLRGPQVDVKGPFVEAEVPDVDLECPDAKLKGPKFKMPEMHFKAPKISMPDVDLHLKGPKVKGDADVSVPKLEGDLTGPSVGVEVPDVELECPDAKLKGPKFKMPDMHFKAPKISMPDVDLHLKGPKVKGDVDVSVPKLEGDLTGPSVGVEVPDVELECPDAKLKGPKFKMPEMHFKTPKISMPDVDLHLKGPKVKGDMDVSVPKVEGEMKVPDVDIKGPKMDIDAPDVDVHGPDWHLKMPKMKMPKFSMPGFKAEGPEVDVNLPKADVVVSGPKVDVEVPDVSLEGPEGKLKGPKLKMPEMHFKAPKISMPDVDLHLKGPKVKGDVDVSLPKLEGDLTGPSVDVEVPDVELECPDAKLKGPKFKMPEMHFKTPKISMPDVNLNLKGPKVKGDMDVSVPKVEGEMKVPDVDIRGPKVDIDAPDVDVHGPDWHLKMPKMKMPKFSMPGFKGEGPEVDVNLPKADVDVSGPKVDVEVPDVSLEGPEGKLKGPKFKMPEMHFKTPKISMPDVDFNLKGPKIKGDVDVSAPKLEGELKGPELDVKGPKLDADMPEVAVEGPNGKWKTPKFKMPDMHFKAPKISMPDLDLHLKSPKAKGEVDVDVPKLEGDLKGPHVDVSGPDIDIEGPEGKLKGPKFKMPDMHFKAPNISMPDVDLNLKGPKIKGDVDVSVPEVEGKLEVPDMNIRGPKVDVNAPDVQAPDWHLKMPKMKMPKFSMPGFKAEGPEVDVNLPKADVDISGPKVDIEGPDVNIEGPEGKLKGPKLKMPEMNIKAPKISMPDFDLHLKGPKVKGDVDVSLPKVEGDLKGPEVDIKGPKVDINAPDVGVQGPDWHLKMPKVKMPKFSMPGFKGEGPDGDVKLPKADIDVSGPKVDIEGPDVNIEGPEGKLKGPKFKMPEMNIKAPKISMPDIDLNLKGPKVKGDVDVSLPKVEGDLKGPEVDIKGPKVDIDAPDVDVHGPDWHLKMPKIKMPKISMPGFKGEGPDVDVNLPKADIDVSGPKVDVECPDVNIEGPEGKWKSPKFKMPEMHFKTPKISMPDIDLNLTGPKIKGDVDVTGPKVEGDLKGPEVDLKGPKVDIDVPDVNVQGPDWHLKMPKMKMPKFSMPGFKAEGPEVDVNLPKADVDVSGPKVDVEGPDVNIEGPEGKLKGPKFKMPEMNIKAPKIPMPDFDLHLKGPKVKGDVDISLPKVEGDLKGPEVDIRGPQVDIDVPDVGVQGPDWHLKMPKVKMPKFSMPGFKGEGPDVDVNLPKADLDVSGPKVDIDVPDVNIEGPEGKLKGPKFKMPEMNIKAPKISMPDIDLNLKGPKVKGDMDVSLPKVEGDMKVPDVDIKGPKVDINAPDVDVQGPDWHLKMPKIKMPKISMPGFKGEGPEVDVNLPKADLDVSGPKVDVDVPDVNIEGPDAKLKGPKFKMPEMNIKAPKISMPDLDLNLKGPKMKGEVDVSLANVEGDLKGPALDIKGPKIDVDAPDIDIHGPDAKLKGPKLKMPDMHVNMPKISMPEIDLNLKGSKLKGDVDVSGPKLEGDIKAPSLDIKGPEVDVSGPKLNIEGKSKKSRFKLPKFNFSGSKVQTPEVDVKGKKPDIDITGPKVDINAPDVEVQGKVKGSKFKMPFLSISSPKVSMPDVELNLKSPKVKGDLDIAGPNLEGDFKGPKVDIKAPEVNLNAPDVDVHGPDWNLKMPKMKMPKFSVSGLKAEGPDVAVDLPKGDINIEGPSMNIEGPDLNVEGPEGGLKGPKFKMPDMNIKAPKISMPDIDLNLKGPKVKGDVDISLPKLEGDLKGPEVDIKGPKVDINAPDVDVHGPDWHLKMPKVKMPKFSMPGFKGEGPEVDVTLPKADIDISGPNVDVDVPDVNIEGPDAKLKGPKFKMPEMNIKAPKISMPDFDLNLKGPKMKGDVVVSLPKVEGDLKGPEVDIKGPKVDIDTPDINIEGSEGKFKGPKFKIPEMHLKAPKISMPDIDLNLKGPKVKGDVDVSLPKMEGDLKGPEVDIKGPKVDINAPDVDVQGPDWHLKMPKVKMPKFSMPGFKGEGPDVDVNLPKADLDVSGPKVDIDVPDVNIEGPEGKLKGPKFKMPEMNIKAPKISMPDIDLNLKGPKVKGDMDVSLPKVEGDMQVPDLDIKGPKVDINAPDVDVRGPDWHLKMPKIKMPKISMPGFKGEGPEVDVNLPKADLDVSGPKVDVDVPDVNIEGPDAKLKGPKFKMPEMNIKAPKISMPDFDLHLKGPKVKGDVDVSLPKMEGDLKAPEVDIKGPKVDIDAPDVDVHGPDWHLKMPKVKMPKFSMPGFKGEGPEVDVNLPKADIDVSGPKVDIDTPDIDIHGPEGKLKGPKFKMPDLHLKAPKISMPEVDLNLKGPKMKGDVDVSLPKVEGDLKGPEVDIKGPKVDIDVPDVDVQGPDWHLKMPKVKMPKFSMPGFKGEGPDVDVNLPKADLDVSGPKVDIDVPDVNIEGPDAKLKGPKFKMPEMNIKAPKISMPDFDLHLKGPKVKGDVDVSLPKVEGDLKGPEVDIKGPKVDIDAPDVDVHGPDWHLKMPKVKMPKFSMPGFKGEGPDVDVTLPKADIEISGPKVDIDAPDVSIEGPDAKLKGPKFKMPEMNIKAPKISMPDIDFNLKGPKVKGDVDVSLPKVEGDLKGPEIDIKGPSLDIDTPDVNIEGPEGKLKGPKFKMPEMNIKAPKISMPDFDLHLKGPKVKGDVDVSLPKVESDLKGPEVDIEGPEGKLKGPKFKMPDVHFKSPQISMSDIDLNLKGPKIKGDMDISVPKLEGDLKGPKVDVKGPKVGIDTPDIDIHGPEGKLKGPKFKMPDLHLKAPKISMPEVDLNLKGPKVKGDMDISLPKVEGDLKGPEVDIRDPKVDIDVPDVDVQGPDWHLKMPKVKMPKFSMPGFKGEGPDVDVNLPKADIDVSGPKVDVDVPDVNIEGPDAKLKGPKFKMPEMSIKAPKISMPDIDLNLKGPKVKGDVDVTLPKVEGDLKGPEADIKGPKVDINTPDVDVHGPDWHLKMPKVKMPKFSMPGFKGEGPDVDVSLPKADIDVSGPKVDVDIPDVNIEGPDAKLKGPKFKMPEINIKAPKISIPDVDLDLKGPKVKGDFDVSVPKVEGTLKGPEVDLKGPRLDFEGPDAKLSGPSLKMPSLEISAPKVTAPDVDLHLKAPKIGFSGPKLEGGEVDLKGPKVEAPSLDVHMDSPDINIEGPDVKIPKFKKPKFGFGAKSPKADIKSPSLDVTVPEAELNLETPEISVGGKGKKSKFKMPKIHMSGPKIKAKKQGFDLNVPGGEIDASLKAPDVDVNIAGPDAALKVDVKSPKTKKTMFGKMYFPDVEFDIKSPKFKAEAPLPSPKLEGELQAPDLELSLPAIHVEGLDIKAKAPKVKMPDVDISVPKIEGDLKGPKVQANLGAPDINIEGLDAKVKTPSFGISAPQVSIPDVNVNLKGPKIKGDVPSVGLEGPDVDLQGPEAKIKFPKFSMPKIGIPGVKMEGGGAEVHAQLPSLEGDLRGPDVKLEGPDVSLKGPGVDLPSVNLSMPKVSGPDLDLNLKGPSLKGDLDASVPSMKVHAPGLNLSGVGGKMQVGGDGVKVPGIDATTKLNVGAPDVTLRGPSLQGDLAVSGDIKCPKVSVGAPDLSLEASEGSIKLPKMKLPQFGISTPGSDLHVNAKGPQVSGELKGPGVDVNLKGPRISAPNVDFNLEGPKVKGSLGATGEIKGPTVGGGLPGIGVQGLEGNLQMPGIKSSGCDVNLPGVNVKLPTGQISGPEIKGGLKGSEVGFHGAAPDISVKGPAFNMASPESDFGINLKGPKIKGGADVSGGVSAPDISLGEGHLSVKGSGGEWKGPQVSSALNLDTSKFAGGLHFSGPKVEGGVKGGQIGLQAPGLSVSGPQGHLESGSGKVTFPKMKIPKFTFSGRELVGREMGVDVHFPKAEASIQAGAGDGEWEESEVKLKKSKIKMPKFNFSKPKGKGGVTGSPEASISGSKGDLKSSKASLGSLEGEAEAEASSPKGKFSLFKSKKPRHRSNSFSDEREFSGPSTPTGTLEFEGGEVSLEGGKVKGKHGKLKFGTFGGLGSKSKGHYEVTGSDDETGKLQGSGVSLASKKSRLSSSSSNDSGNKVGIQLPEVELSVSTKKE

SEQ ID NO: 38 - protein, 1894 aa, Homo sapiens
MAPEAGATLRAPRRLSWAALLLLAALLPVASSAAASVDHPLKPRHVKLLSTKMGLKVTWDPPKDATSRPVEHYNIAYGKSLKSLKYIKVNAETYSFLIEDVEPGVVYFVLLTAENHSGVSRPVYRAESPPGGEWIEIDGFPIKGPGPFNETVTEKEVPNKPLRVRVRSSDDRLSVAWKAPRLSGAKSPRRSRGFLLGYGESGRKMNYVPLTRDERTHEIKKLASESVYVVSLQSMNSQGRSQPVYRAALTKRKISEEDELDVPDDISVRVMSSQSVLVSWVDPVLEKQKKVVASRQYTVRYREKGELARWDYKQIANRRVLIENLIPDTVYEFAVRISQGERDGKWSTSVFQRTPESAPTTAPENLNVWPVNGKPTVVAASWDALPETEGKVKEYILSYAPALKPFGAKSLTYPGDTTSALVDGLQPGERYLFKIRATNRRGLGPHSKAFIVAMPTTSKADVEQNTEDNGKPEKPEPSSPSPRAPASSQHPSVPASPQGRNAKDLLLDLKNKILANGGAPRKPQLRAKKAEELDLQSTEITGEEELGSREDSPMSPSDTQDQKRTLRPPSRHGHSVVAPGRTAVRARMPALPRREGVDKPGFSLATQPRPGAPPSASASPAHHASTQGTSHRPSLPASLNDNDLVDSDEDERAVGSLHPKGAFAQPRPALSPSRQSPSSVLRDRSSVHPGAKPASPARRTPHSGAAEEDSSASAPPSRLSPPHGGSSRLLPTQPHLSSPLSKGGKDGEDAPATNSNAPSRSTMSSSVSSHLSSRTQVSEGAEASDGESHGDGDREDGGRQAEATAQTLRARPASGHFHLLRHKPFAANGRSPSRFSIGRGPRLQPSSSPQSTVPSRAHPRVPSHSDSHPKLSSGIHGDEEDEKPLPATVVNDHVPSSSRQPISRGWEDLRRSPQRGASLHRKEPIPENPKSTGADTHPQGKYSSLASKAQDVQQSTDADTEGHSPKAQPGSTDRHASPARPPAARSQQHPSVPRRMTPGRAPQQQPPPPVATSQHHPGPQSRDAGRSPSQPRLSLTQAGRPRPTSQGRSHSSSDPYTASSRGMLPTALQNQDEDAQGSYDDDSTEVEAQDVRAPAHAARAKEAAASLPKHQQVESPTGAGAGGDHRSQRGHAASPARPSRPGGPQSRARVPSRAAPGKSEPPSKRPLSSKSQQSVSAEDDEEEDAGFFKGGKEDLLSSSVPKWPSSSTPRGGKDADGSLAKEEREPAIALAPRGGSLAPVKRPLPPPPGSSPRASHVPSRLPPRSAATVSPVAGTHPWPQYTTRAPPGHFSTTPMLSLRQRMMHARFRNPLSRQPARPSYRQGYNGRPNVEGKVLPGSNGKPNGQRIINGPQGTKWVVDLDRGLVLNAEGRYLQDSHGNPLRIKLGGDGRTIVDLEGTPVVSPDGLPLFGQGRHGTPLANAQDKPILSLGGKPLVGLEVIKKTTHPPTTTMQPTTTTTPLPTTTTPRPTTATTRRTTTTRRTTTRRPTTTVRTTTRTTTTTTPTPTTPIPTCPPGTLERHDDDGNLIMSSNGIPECYAEEDEFSGLETDTAVPTEEAYVIYDEDYEFETSRPPTTTEPSTTATTPRVIPEEGAISSFPEEEFDLAGRKRFVAPYVTYLNKDPSAPCSLTDALDHFQVDSLDEIIPNDLKKSDLPPQHAPRNITVVAVEGCHSFVIVDWDKATPGDVVTGYLVYSASYEDFIRNKWSTQASSVTHLPIENLKPNTRYYFKVQAQNPHGYGPISPSVSFVTESDNPLLVVRPPGGEPIWIPFAFKHDPSYTDCHGRQYVKRTWYRKFVGVVLCNSLRYKIYLSDNLKDTFYSIGDSWGRGEDHCQFVDSHLDGRTGPQSYVEALPTIQGYYRQYRQEPVRFGNIGFGTPYYYVGWYECGVSIPGKW

SEQ ID NO: 39 - protein, 198 aa, Homo sapiens
MAADISESSGADCKGDPRNSAKLDADYPLRVLYCGVCSLPTEYCEYMPDVAKCRQWLEKNFPNEFAKLTVENSPKQEAGISEGQGTAGEEEEKKKQKRGGRGQIKQKKKTVPQKVTIAKIPRAKKKYVTRVCGLATFEIDLKEAQRFFAQKFSCGASVTGEDEIIIQGDFTDDIIDVIQEKWPEVDDDSIEDLGEVKK

SEQ ID NO: 40 - protein, 267 aa, Homo sapiens
MARFLTLCTWLLLLGPGLLATVRAECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 41 - protein, 105 aa, Homo sapiens
MAKISSPTETERCIESLIAVFQKYAGKDGYNYTLSKTEFLSFMNTELAAFTKNQKDPGVLDRMMKKLDTNSDGQLDFSEFLNLIGGLAMACHDSFLKAVPSQKRT

SEQ ID NO: 42 - protein, 159 aa, Homo sapiens [sequence omitted]

SEQ ID NO: 43 - protein, 136 aa, Homo sapiens [sequence omitted]

SEQ ID NO: 44 - protein, 257 aa, Homo sapiens [sequence omitted]

SEQ ID NO: 45 - protein, 221 aa, Homo sapiens
MSYGRPPPDVEGMTSLKVDNLTYRTSPDTLRRVFEKYGRVGDVYIPRDRYTKESRGFAFVRFHDKRDAEDAMDAMDGAVLDGRELRVQMARYGRPPDSHHSRRGPPPRRYGGGGYGRRSRSPRRRRRSRSRSRSRSRSRSRSRYSRSKSRSRTRSRSRSTSKSRSARRSKSKSSSVSRSRSRSRSRSRSRSPPPVSKRESKSRSRSKSPPKSPEEEGAVSS

SEQ ID NO: 46 - protein, 424 aa, Homo sapiens
MAAGPISERNQDATVYVGGLDEKVSEPLLWELFLQAGPVVNTHMPKDRVTGQHQGYGFVEFLSEEDADYAIKIMNMIKLYGKPIRVNKASAHNKNLDVGANIFIGNLDPEIDEKLLYDTFSAFGVILQTPKIMRDPDTGNSKGYAFINFASFDASDAAIEAMNGQYLCNRPITVSYAFKKDSKGERHGSAAERLLAAQNPLSQADRPHQLFADAPPPPSAPNPVVSSLGSGLPPPGMPPPGSFPPPVPPPGALPPGIPPAMPPPPMPPGAAGHGPPSAGTPGAGHPGHGHSHPHPFPPGGMPHPGMSQMQLAHHGPHGLGHPHAGPPGSGGQPPPRPPPGMPHPGPPPMGMPPRGPPFGSPMGHPGPMPPHGMRGPPPLMPPHGYTGPPRPPPYGYQRGPLPPPRPTPRPPVPPRGPLRGPLPQ

SEQ ID NO: 47 - protein, 590 aa, Homo sapiens
MHLARLVGSCSLLLLLGALSGWAASDDPIEKVIEGINRGLSNAEREVGKALDGINSGITHAGREVEKVFNGLSNMGSHTGKELDKGVQGLNHGMDKVAHEINHGIGQAGKEAEKLGHGVNNAAGQVGKEADKLIHHGVHHGANQAGSEAGKFGQGVDNAAGQAGNEAGRFGQGVHHAAGQAGNEAGRFGQGVHHAAGQAGNEAGRFGQGAHHGLSEGWKETEKFGQGIHHAAGQVGKEAEKFGQGAHHAAGQAGNEAGRFGQGVHHGLSEGWKETEKFGQGVHHTAGQVGKEAEKFGQGAHHAAGQAGNEAGRFGQGAHHAAGQAGNEAGRFGQGVHHGLSEGWKETEKFGQGVHHAASQFGKETEKLGHGVHHGVNEAWKEAEKFGQGVHHAASQVGKEEDRVVQGLHHGVSQAGREAGQFGHDIHHTAGQAGKEGDIAVHGVQPGVHEAGKEAGQFGQGVHHTLEQAGKEADKAVQGFHTGVHQAGKEAEKLGQGVNHAADQAGKEVEKLGQGAHHAAGQAGKELQNAHNGVNQASKEANQLLNGNHQSGSSSHQGGATTTPLASGASVNTPFINLPALWRSVANIMP

SEQ ID NO: 48 - protein, 379 aa, Homo sapiens
MVFFTCNACGESVKKIQVEKHVSVCRNCECLSCIDCGKDFWGDDYKNHVKCISEDQKYGGKGYEGKTHKGDIKQQAWIQKISELIKRPNVSPKVRELLEQISAFDNVPRKKAKFQNWMKNSLKVHNESILDQVWNIFSEASNSEPVNKEQDQRPLHPVANPHAEISTKVPASKVKDAVEQQGEVKKNKRERKEERQKKRKREKKELKLENHQENSRNQKPKKRKKGQEADLEAGGEEVPEANGSAGKRSKKKKQRKDSASEEEAHVGAGKRKRRHSEVETDSKKKKMKLPEHPEGGEPEDDEAPAKGKFNWKGTIKAILKQAPDNEITIKKLRKKVLAQYYTVTDEHHRSEEELLVIFNKKISKNPTFKLLKDKVKLVK

SEQ ID NO: 49 - protein, 331 aa, Homo sapiens
MGGEQEEERFDGMLLAMAQQHEGGVQELVNTFFSFLRRKTDFFIGGEEGMAEKLITQTFSHHNQLAQKTRREKRARQEAERREKAERAARLAKEAKSETSGPQIKELTDEEAERLQLEIDQKKDAENHEAQLKNGSLDSPGKQDTEEDEEEDEKDKGKLKPNLGNGADLPNYRWTQTLSELDLAVPFCVNFRLKGKDMVVDIQRRHLRVGLKGQPAIIDGELYNEVKVEESSWLIEDGKVVTVHLEKINKMEWWSRLVSSDPEINTKKINPENSKLSDLDSETRSMVEKMMYDQRQKSMGLPTSDEQKKQEILKKFMDQHPEMDFSKAKFN

SEQ ID NO: 50 - protein, 422 aa, Homo sapiens
MPAGRRGPAAQSARRPPPLLPLLLLLCVLGAPRAGSGAHTAVISPQDPTLLIGSSLLATCSVHGDPPGATAEGLYWTLNGRRLPPELSRVLNASTLALALANLNGSRQRSGDNLVCHARDGSILAGSCLYVGLPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGACEPRGGEPSSGPVRRELKQFLGWLKKHAYCSNLSFRLYDQWRAWMQKSHKTRNQDEGILPSGRRGTARGPAR

SEQ ID NO: 51 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 52 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 53 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 54 - protein, 509 aa, Homo sapiens
MQSWSRVYCSLAKRGHFNRISHGLQGLSAVPLRTYADQPIDADVTVIGSGPGGYVAAIKAAQLGFKTVCIEKNETLGGTCLNVGCIPSKALLNNSHYYHMAHGKDFASRGIEMSEVRLNLDKMMEQKSTAVKALTGGIAHLFKQNKVVHVNGYGKITGKNQVTATKADGGTQVIDTKNILIATGSEVTPFPGITIDEDTIVSSTGALSLKKVPEKMVVIGAGVIGVELGSVWQRLGADVTAVEFLGHVGGVGIDMEISKNFQRILQKQGFKFKLNTKVTGATKKSDGKIDVSIEAASGGKAEVITCDVLLVCIGRRPFTKNLGLEELGIELDPRGRIPVNTRFQTKIPNIYAIGDVVAGPMLAHKAEDEGIICVEGMAGGAVHIDYNCVPSVIYTHPEVAWVGKSEEQLKEEGIEYKVGKFPFAANSRAKTNADTDGMVKILGQKSTDRVLGAHILGPGAGEMVNEAALALEYGASCEDIARVCHAHPTLSEAFREANLAASFGKSINF

SEQ ID NO: 55 - protein, 1005 aa, Homo sapiens
MTTEVGSVSEVKKDSSQLGTDATKEKPKEVAENQQNQSSDPEEEKGSQPPPAAESQSSLRRQKREKETSESRGISRFIPPWLKKQKSYTLVVAKDGGDKKEPTQAVVEEQVLDKEEPLPEEQRQAKGDAEEMAQKKQEIKVEVKEEKPSVSKEEKPSVSKVEMQPTELVSKEREEKVKETQEDKLEGGAAKRETKEVQTNELKAEKASQKVTKKTKTVQCKVTLLDGTEYSCDLEKHAKGQVLFDKVCEHLNLLEKDYFGLLFQESPEQKNWLDPAKEIKRQLRNLPWLFTFNVKFYPPDPSQLTEDITRYFLCLQLRQDIASGRLPCSFVTHALLGSYTLQAELGDYDPEEHGSIDLSEFQFAPTQTKELEEKVAELHKTHRGLSPAQADSQFLENAKRLSMYGVDLHHAKDSEGVDIKLGVCANGLLIYKDRLRINRFAWPKILKISYKRSNFYIKVRPAELEQFESTIGFKLPNHRAAKRLWKVCVEHHTFYRLVSPEQPPKAKFLTLGSKFRYSGRTQAQTRQASTLIDRPAPHFERTSSKRVSRSLDGAPIGVMDQSLMKDFPGAAGEISAYGPGLVSIAVVQDGDGRREVRSPTKAPHLQLIEGKKNSLRVEGDNIYVRHSNLMLEELDKAQEDILKHQASISELKRNFMESTPEPRPNEWEKRRITPLSLQTQGSSHETLNIVEEKKRAEVGKDERVITEEMNGKEISPGSGPGEIRKVEPVTQKDSTSLSSESSSSSSESEEEDVGEYRPHHRVTEGTIREEQEYEEEVEEEPRPAAKVVEREEAVPEASPVTQAGASVITVETVIQENVGAQKIPGEKSVHEGALKQDMGEEAEEEPQKVNGEVSHVDIDVLPQIICCSEPPVVKTEMVTISDASQRTEISTKEVPIVQTETKTITYESPQIDGGAGGDSGTLLTAQTITSESVSTTTTTHITKTVKGGISETRIEKRIVITGDGDIDHDQALAQAIREAREQHPDMSVTRVVVHKETELAEEGED

SEQ ID NO: 56 - protein, 219 aa, Homo sapiens
MLRAGAPTGDLPRAGEVHTGTTIMAVEFDGGVVMGSDSRVSAGEAVVNRVFDKLSPLHERIYCALSGSAADAQAVADMAAYQLELHGIELEEPPLVLAAANVVRNISYKYREDLSAHLMVAGWDQREGGQVYGTLGGMLTRQPFAIGGSGSTFIYGYVDAAYKPGMSPEECRRFTTDAIALAMSRDGSSGGVIYLVTITAAGVDHRVILGNELPKFYDE

SEQ ID NO: 57 - protein, 151 aa, Homo sapiens [sequence omitted]

SEQ ID NO: 58 - protein, 266 aa, Homo sapiens [sequence omitted]

SEQ ID NO: 59 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGSSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 60 - protein, 1214 aa, Homo sapiens
MAVRKKDGGPNVKYYEAADTVTQFDNVRLWLGKNYKKYIQAEPPTNKSLSSLVVQLLQFQEEVFGKHVSNAPLTKLPIKCFLDFKAGGSLCHILAAAYKFKSDQGWRRYDFQNPSRMDRNVEMFMTIEKSLVQNNCLSRPNIFLCPEIEPKLLGKLKDIIKRHQGTVTEDKNNASHVVYPVPGNLEEEEWVRPVMKRDKQVLLHWGYYPDSYDTWIPASEIEASVEDAPTPEKPRKVHAKWILDTDTFNEWMNEEDYEVNDDKNPVSRRKKISAKTLTDEVNSPDSDRRDKKGGNYKKRKRSPSPSPTPEAKKKNAKKGPSTPYTKSKRGHREEEQEDLTKDMDEPSPVPNVEEVTLPKTVNTKKDSESAPVKGGTMTDLDEQEDESMETTGKDEDENSTGNKGEQTKNPDLHEDNVTEQTHHIIIPSYAAWFDYNSVHAIERRALPEFFNGKNKSKTPEIYLAYRNFMIDTYRLNPQEYLTSTACRRNLAGDVCAIMRVHAFLEQWGLINYQVDAESRPTPMGPPPTSHFHVLADTPSGLVPLQPKTPQQTSASQQMLNFPDKGKEKPTDMQNFGLRTDMYTKKNVPSKSKAAASATREWTEQETLLLLEALEMYKDDWNKVSEHVGSRTQDECILHFLRLPIEDPYLEDSEASLGPLAYQPIPFSQSGNPVMSTVAFLASVVDPRVASAAAKSALEEFSKMKEEVPTALVEAHVRKVEEAAKVTGKADPAFGLESSGIAGTTSDEPERIEESGNDEARVEGQATDEKKEPKEPREGGGAIEEEAKEKTSEAPKKDEEKGKEGDSEKESEKSDGDPIVDPEKEKEPKEGQEEVLKEVVESEGERKTKVERDIGEGNLSTAAAAALAAAAVKAKHLAAVEERKIKSLVALLVETQMKKLEIKLRHFEELETIMDREREALEYQRQQLLADRQAFHMEQLKYAEMRARQQHFQQMHQQQQQPPPALPPGSQPIPPTGAAGPPAVHGLAVAPASVVPAPAGSGAPPGSLGPSEQIGQAGSTAGPQQQQPAGAPQPGAVPPGVPPPGPHGPSPFPNQQTPPSMMPGAVPGSGHPGVAGNAPLGLPFGMPPPPPPPAPSIIPFGSLADSISINLPAPPNLHGHHHHLPFAPGTLPPPNLPVSMANPLHPNLPATTTMPSSLPLGPGLGSAAAQSPAIVAAVQGNLLPSASPLPDPGTPLPPDPTAPSPGTVTPVPPPQ

SEQ ID NO: 61 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 62 - protein, 539 aa, Homo sapiens
MPENVAPRSGATAGAAGGRGKGAYQDRDKPAQIRFSNISAAKAVADAIRTSLGPKGMDKMIQDGKGDVTITNDGATILKQMQVLHPAARMLVELSKAQDIEAGDGTTSVVIIAGSLLDSCTKLLQKGIHPTIISESFQKALEKGIEILTDMSRPVELSDRETLLNSATTSLNSKVVSQYSSLLSPMSVNAVMKVIDPATATSVDLRDIKIVKKLGGTIDDCELVEGLVLTQKVSNSGITRVEKAKIGLIQFCLSAPKTDMDNQIVVSDYAQMDRVLREERAYILNLVKQIKKTGCNVLLIQKSILRDALSDLALHFLNKMKIMVIKDIEREDIEFICKTIGTKPVAHIDQFTADMLGSAELAEEVNLNGSGKLLKITGCASPGKTVTIVVRGSNKLVIEEAERSIHDALCVIRCLVKKRALIAGGGAPEIELALRLTEYSRTLSGMESYCVRAFADAMEVIPSTLAENAGLNPISTVTELRNRHAQGEKTAGINVRKGGISNILEELVVQPLLVSVSALTLATETVRSILKIDDVVNTR

SEQ ID NO: 63 - protein, 758 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPETNRKTDDPEQKAKVKKKKPKLLNKFDKTIKAELDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDPGTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETKTWEEGKVLIFDDSFEHEVWQDASSFRLIFIVDVWHPELTPQQRRSLPAI

SEQ ID NO: 64 - protein, 491 aa, Homo sapiens
MPLPVQVFNLQGAVEPMQIDVDPQEDPQNAPDVNYVVENPSLDLEQYAASYSGLMRIERLQFIADHCPTLRVEALKMALSFVQRTFNVDMYEEIHRKLSEATRSSLRELQNAPDAIPESGVEPPALDTAWVEATRKKALLKLEKLDTDLKNYKGNSIKESIRRGHDDLGDHYLDCGDLSNALKCYSRARDYCTSAKHVINMCLNVIKVSVYLQNWSHVLSYVSKAESTPEIAEQRGERDSQTQAILTKLKCAAGLAELAARKYKQAAKCLLLASFDHCDFPELLSPSNVAIYGGLCALATFDRQELQRNVISSSSFKLFLELEPQVRDIIFKFYESKYASCLKMLDEMKDNLLLDMYLAPHVRTLYTQIRNRALIQYFSPYVSADMHRMAAAFNTTVAALEDELTQLILEGLISARVDSHSKILYARDVDQRSTTFEKSLLMGKEFQRRAKAMMLRAAVLRNQIHVKSPPREGSQGELTPANSQSRMSTNM

SEQ ID NO: 65 - protein, 1512 aa, Homo sapiens
MPSSSDTALGGGGGLSWAEKKLEERRKRRRFLSPQQPPLLLPLLQPQLLQPPPPPPPLLFLAAPGTAAAAAAAAAASSSCFSPGPPLEVKRLARGKRRAGGRQKRRRGPRAGQEAEKRRVFSLPQPQQDGGGGASSGGGVTPLVEYEDVSSQSEQGLLLGGASAATAATAAGGTGGSGGSPASSSGTQRRGEGSERRPRRDRRSSSGRSKERHREHRRRDGQRGGSEASKSRSRHSHSGEERAEVAKSGSSSSSGGRRKSASATSSSSSSRKDRDSKAHRSRTKSSKEPPSAYKEPPKAYREDKTEPKAYRRRRSLSPLGGRDDSPVSHRASQSLRSRKSPSPAGGGSSPYSRRLPRSPSPYSRRRSPSYSRHSSYERGGDVSPSPYSSSSWRRSRSPYSPVLRRSGKSRSRSPYSSRHSRSRSRHRLSRSRSRHSSISPSTLTLKSSLAAELNKNKKARAAEAARAAEAAKAAEATKAAEAAAKAAKASNTSTPTKGNTETSASASQTNHVKDVKKIKIEHAPSPSSGGTLKNDKAKTKPPLQVTKVENNLIVDKATKKAVIVGKESKSAATKEESVSLKEKTKPLTPSIGAKEKEQHVALVTSTLPPLPLPPMLPEDKEADSLRGNISVKAVKKEVEKKLRCLLADLPLPPELPGGDDLSKSPEEKKTATQLHSKRRPKICGPRYGETKEKDIDWGKRCVDKFDIIGIIGEGTYGQVYKARDKDTGEMVALKKVRLDNEKEGFPITAIREIKILRQLTHQSIINMKEIVTDKEDALDFKKDKGAFYLVFEYMDHDLMGLLESGLVHFNENHIKSFMRQLMEGLDYCHKKNFLHRDIKCSNILLNNRGQIKLADFGLARLYSSEESRPYTNKVITLWYRPPELLLGEERYTPAIDVWSCGCILGELFTKKPIFQANQELAQLELISRICGSPCPAVWPDVIKLPYFNTMKPKKQYRRKLREEFVFIPAAALDLFDYMLALDPSKRCTAEQALQCEFLRDVEPSKMPPPDLPLWQDCHELWSKKRRRQKQMGMTDDVSTIKAPRKDLSLGLDDSRTNTPQGVLPSSQLKSQGSSNVAPVKTGPGQHLNHSELAILLNLLQSKTSVNMADFVQVLNIKVNSETQQQLNKINLPAGILATGEKQTDPSTPQQESSKPLGGIQPSSQTIQPKVETDAAQAAVQSAFAVLLTQLIKAQQSKQKDVLLEERENGSGHEASLQLRPPPEPSTPVSGQDDLIQHQDMRILELTPEPDRPRILPPDQRPPEPPEPPPVTEEDLDYRTENQHVPTTSSSLTDPHAGVKAALLQLLAQHQPQDDPKREGGIDYQAGDTYVSTSDYKDNFGSSSFSSAPYVSNDGLGSSSAPPLERRSFIGNSDIQSLDNYSTASSHSGGPPQPSAFSESFPSSVAGYGDIYLNAGPMLFSGDKDHRFEYSHGPIAVLANSSDPSTGPESTHPLPAKMHNYNYGGNLQENPSGPSLMHGQTWTSPAQGPGYSQGYRGHISTSTGRGRGRGLPY

SEQ ID NO: 66 - protein, 352 aa, Homo sapiens
MSVAGLKKQFHKATQKVSEKVGGAEGTKLDDDFKEMERKVDVTSRAVMEIMTKTIEYLQPNPASRAKLSMINTMSKIRGQEKGPGYPQAEALLAEAMLKFGRELGDDCNFGPALGEVGEAMRELSEVKDSLDIEVKQNFIDPLQNLHDKDLREIQHHLKKLEGRRLDFDYKKKRQGKIPDEELRQALEKFDESKEIAESSMFNLLEMDIEQVSQLSALVQAQLEYHKQAVQILQQVTVRLEERIRQASSQPRREYQPKPRMSLEFPTGDSTQPNGGLSHTGTPKPSGVQMDQPCCRALYDFEPENEGELGFKEGDIITLTNQIDENWYEGMLHGHSGFFPINYVEILVALPH

SEQ ID NO: 67 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 68 - protein, 318 aa, Homo sapiens
MPKRGKKGAVAEDGDELRTEPEAKKSKTAAKKNDKEAAGEGPALYEDPPDQKTSPSGKPATLKICSWNVDGLRAWIKKKGLDWVKEEAPDILCLQETKCSENKLPAELQELPGLSHQYWSAPSDKEGYSGVGLLSRQCPLKVSYGIGDEEHDQEGRVIVAEFDSFVLVTAYVPNAGRGLVRLEYRQRWDEAFRKFLKGLASRKPLVLCGDLNVAHEEIDLRNPKGNKKNAGFTPQERQGFGELLQAVPLADSFRHLYPNTPYAYTFWTYMMNARSKNVGWRLDYFLLSHSLLPALCDSKIRSKALGSDHCPITLYLAL

SEQ ID NO: 69 - protein, 263 aa, Homo sapiens
MALASVLERPLPVNQRGFFGLGGRADLLDLGPGSLSDGLSLAAPGWGVPEEPGIEMLHGTTTLAFKFRHGVIVAADSRATAGAYIASQTVKKVIEINPYLLGTMAGGAADCSFWERLLARQCRIYELRNKERISVAAASKLLANMVYQYKGMGLSMGTMICGWDKRGPGLYYVDSEGNRISGATFSVGSGSVYAYGVMDRGYSYDLEVEQAYDLARRAIYQATYRDAYSGGAVNLYHVREDGWIRVSSDNVADLHEKYSGSTP

SEQ ID NO: 70 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 71 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 72 - protein, 208 aa, Homo sapiens [sequence omitted]

SEQ ID NO: 73 - protein, 290 aa, Homo sapiens
MAPKRQSPLPPQKKKPRPPPALGPEETSASAGLPKKGEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 74 - protein, 152 aa, Homo sapiens [sequence omitted]

SEQ ID NO: 75 - protein, 166 aa, Homo sapiens
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 76 - protein, 476 aa, Homo sapiens
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGGSSGGSSGGSSGNSGGSRGDSGSESSWGSSTGSSSGNHGGSGGGNGHKPGCEKPGNEARGSGESGIQNSETSPGMFNFDTFWKNFKSKLGFINWDAINKNQVPPPSTRALLYFSRLWEDFKQNTPFLNWKAIIEGADASSLQKRAGRDDQNYNYNQHAYPTAYGGKYSVKTPAKGGVSPSSSASRVQPGLLQWVKFW

SEQ ID NO: 77 - protein, 128 aa, Homo sapiens [sequence omitted]

SEQ ID NO: 78 - protein, 288 aa, Homo sapiens [sequence omitted]

SEQ ID NO: 79 - protein, 582 aa, Homo sapiens
MFPAGPPSHSLLRLPLLQLLLLVVQAVGRGLGRASPAGGPLEDVVIERYHIPRACPREVQMGDFVRYHYNGTFEDGKKFDSSYDRNTLVAIVVGVGRLITGMDRGLMGMCVNERRRLIVPPHLGYGSIGLAGLIPPDATLYFDVVLLDVWNKEDTVQVSTLLRPPHCPRMVQDGDFVRYHYNGTLLDGTSFDTSYSKGGTYDTYVGSGWLIKGMDQGLLGMCPGERRKIIIPPFLAYGEKGYGTVIPPQASLVFHVLLIDVHNPKDAVQLETLELPPGCVRRAGAGDFMRYHYNGSLMDGTLFDSSYSRNHTYNTYIGQGYIIPGMDQGLQGACMGERRRITIPPHLAYGENGTGDKIPGSAVLIFNVHVIDFHNPADVVEIRTLSRPSETCNETTKLGDFVRYHYNCSLLDGTQLFTSHDYGAPQEATLGANKVIEGLDTGLQGMCVGERRQLIVPPHLAHGESGARGVPGSAVLLFEVELVSREDGLPTGYLFVWHKDPPANLFEDMDLNKDGEVPPEEFSTFIKAQVSEGKGRLMPGQDPEKTIGDMFQNQDRNQDGKITVDELKLKSDEDEERVHEEL

SEQ ID NO: 80 - protein, 410 aa, Homo sapiens
MVLSQRQRDELNRAIADYLRSNGYEEAYSVFKKEAELDVNEELDKKYAGLLEKKWTSVIRLQKKVMELESKLNEAKEEFTSGGPLGQKRDPKEWIPRPPEKYALSGHRSPVTRVIFHPVFSVMVSASEDATIKVWDYETGDFERTLKGHTDSVQDISFDHSGKLLASCSADMTIKLWDFQGFECIRTMHGHDHNVSSVAIMPNGDHIVSASRDKTIKMWEVQTGYCVKTFTGHREWVRMVRPNQDGTLIASCSNDQTVRVWVVATKECKAELREHEHVVECISWAPESSYSSISEATGSETKKSGKPGPFLLSGSRDKTIKMWDVSTGMCLMTLVGHDNWVRGVLFHSGGKFILSCADDKTLRVWDYKNKRCMKTLNAHEHFVTSLDFHKTAPYVVTGSVDQTVKVWECR

SEQ ID NO: 81 - protein, 98 aa, Homo sapiens
MAAEPLTELEESIETVVTTFFTFARQEGRKDSLSVNEFKELVTQQLPHLLKDVGSLDEKMKSLDVNQDSELKFNEYWRLIGELAKEIRKKKDLKIRKK

SEQ ID NO: 82 - protein, 246 aa, Homo sapiens
MSRGSSAGFDRHITIFSPEGRLYQVEYAFKAINQGGLTSVAVRGKDCAVIVTQKKVPDKLLDSSTVTHLFKITENIGCVMTGMTADSRSQVQRARYEAANWKYKYGYEIPVDMLCKRIADISQVYTQNAEMRPLGCCMILIGIDEEQGPQVYKCDPAGYYCGFKATAAGVKQTESTSFLEKKVKKKFDWTFEQTVETAITCLSTVLSIDFKPSEIEVGVVTVENPKFRILTEAEIDAHLVALAERD

SEQ ID NO: 83 - protein, 402 aa, Homo sapiens [sequence omitted]

SEQ ID NO: 84 - protein, 709 aa, Homo sapiens
MPSATSHSGSGSKSSGPPPPSGSSGSEAAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLVDPERDMSLRLNEQYEHASIHLWDLLEGKEKPVCGTTYKVLKEIVERVFQSNYFDSTHNHQNGLCEEEEAASAPAVEDQVPEAEPEPAEEYTEQSEVESTEYVNRQFMAETQFTSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSPDQSHQVTGNHQQPPQQNTGFPRSNQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 85 - protein, 437 aa, Homo sapiens
MSVSARSAAAEERSVNSSTMVAQQKNLEGYVGFANLPNQVYRKSVKRGFEFTLMVVGESGLGKSTLINSLFLTDLYSPEYPGPSHRIKKTVQVEQSKVLIKEGGVQLLLTIVDTPGFGDAVDNSNCWQPVIDYIDSKFEDYLNAESRVNRRQMPDNRVQCCLYFIAPSGHGLKPLDIEFMKRLHEKVNIIPLIAKADTLTPEECQQFKKQIMKEIQEHKIKIYEFPETDDEEENKLVKKIKDRLPLAVVGSNTIIEVNGKRVRGRQYPWGVAEVENGEHCDFTILRNMLIRTHMQDLKDVTNNVHYENYRSRKLAAVTYNGVDNNKNKGQLTKSPLAQMEEERREHVAKMKKMEMEMEQVFEMKVKEKVQKLKDSEAELQRRHEQMKKNLEAQHKELEEKRRQFEDEKANWEAQQRILEQQNSSRTLEKNKKKGKIF

SEQ ID NO: 86 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 87 - protein, 863 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 88 - protein, 1461 aa, Homo sapiens
MAAERGARRLLSTPSFWLYCLLLLGRRAPGAAAARSGSAPQSPGASIRTFTPFYFLVEPVDTLSVRGSSVILNCSAYSEPSPKIEWKKDGTFLNLVSDDRRQLLPDGSLFISNVVHSKHNKPDEGYYQCVATVESLGTIISRTAKLIVAGLPRFTSQPEPSSVYAGNNAILNCEVNADLVPFVRWEQNRQPLLLDDRVIKLPSGMLVISNATEGDGGLYRCVVESGGPPKYSDEVELKVLPDPEVISDLVFLKQPSPLVRVIGQDVVLPCVASGLPTPTIKWMKNEEALDTESSERLVLLAGGSLEISDVTEDDAGTYFCIADNGNETIEAQAELTVQAQPEFLKQPTNIYAHESMDIVFECEVTGKPTPTVKWVKNGDMVIPSDYFKIVKEHNLQVLGLVKSDEGFYQCIAENDVGNAQAGAQLIILEHAPATTGPLPSAPRDVVASLVSTRFIKLTWRTPASDPHGDNLTYSVFYTKEGIARERVENTSHPGEMQVTIQNLMPATVYIFRVMAQNKHGSGESSAPLRVETQPEVQLPGPAPNLRAYAASPTSITVTWETPVSGNGEIQNYKLYYMEKGTDKEQDVDVSSHSYTINGLKKYTEYSFRVVAYNKHGPGVSTPDVAVRTLSDVPSAAPQNLSLEVRNSKSIMIHWQPPAPATQNGQITGYKIRYRKASRKSDVTETLVSGTQLSQLIEGLDRGTEYNFRVAALTINGTGPATDWLSAETFESDLDETRVPEVPSSLHVRPLVTSIVVSWTPPENQNIVVRGYAIGYGIGSPHAQTIKVDYKQRYYTIENLDPSSHYVITLKAFNNVGEGIPLYESAVTRPHTDTSEVDLFVINAPYTPVPDPTPMMPPVGVQASILSHDTIRITWADNSLPKHQKITDSRYYTVRWKTNIPANTKYKNANATTLSYLVTGLKPNTLYEFSVMVTKGRRSSTWSMTAHGTTFELVPTSPPKDVTVVSKEGKPKTIIVNWQPPSEANGKITGYIIYYSTDVNAEIHDWVIEPVVGNRLTHQIQELTLDTPYYFKIQARNSKGMGPMSEAVQFRTPKADSSDKMPNDQASGSGGKGSRLPDLGSDYKPPMSGSNSPHGSPTSPLDSNMLLVIIVSVGVITIVVVVIIAVFCTRRTTSHQKKKRAACKSVNGSHKYKGNSKDVKPPDLWIHHERLELKPIDKSPDPNPIMTDTPIPRNSQDITPVDNSMDSNIHQRRNSYRGHESEDSMSTLAGRRGMRPKMMMPFDSQPPQPVISAHPIHSLDNPHHHFHSSSLASPARSHLYHPGSPWPIGTSMSLSDRANSTESVRNTPSTDTMPASSSQTCCTDHQDPEGATSSSYLASSQEEDSGQSLPTAHVRPSHPLKSFAVPAIPPPGPPTYDPALPSTPLLSQQALNHHIHSVKTASIGTLGRSRPPMPVVVPSAPEVQETTRMLEDSESSYEPDELTKEMAHLEGLMKDLNAITTA

SEQ ID NO: 89 - protein, 514 aa, Homo sapiens
MADAEVIILPKKHKKKKERKSLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYSESAKKEVVAEVVKAPQVVAEAAKTAKRKRESESESDETPPAAPQLIKKEKKKSKKDKKAKAGLESGAEPGDGDSDTTKKKKKKKKAKEVELVSE

SEQ ID NO: 90 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 91 - protein, 965 aa, Homo sapiens
MALLCGLGQVTLRIWVSLPSQSENGIGFLAARAFLRSGGMEALTTQLGPGREGSSSPNSKQELQPYSGSSALKPNQVGETSLYGVPIVSLVIDGQERLCLAQISNTLLKNYSYNEIHNRRVALGITCVQCTPVQLEILRRAGAMPISSRRCGMITKREAERLCKSFLGEHKPPKLPENFAFDVVHECAWGSRGSFIPARYNSSRAKCIKCGYCSMYFSPNKFIFHSHRTPDAKYTQPDAANFNSWRRHLKLSDKSATDELSHAWEDVKAMFNGGTRKRTFSLQGGGGGGANGGSGGQGKGGAGGGGGGGPGCGAEMAPGPPPHKSLRCGEDEAAGPPGPPPPHPQRGLGLATGASGPAGPGGPGGGAGVRSYPVIPVPSKGFGLLQKLPPPLFPHPYGFPTAFGLCPKKDDPVLGAGEPKGGPGTGSGGGGAGTGGGAGGPGASHLPPGAGAGPGGGAMFWGHQPSGAAKDAAAVAAAAAAATVYPTFPMFWPAAGSLPVPSYPAAQSQAKAVAAAVAAAAAAAAAAAGSGAPEPLDGAEPAKESGLGAEERCPSALSRGPLDEDGTDEALPPPLAPLPPPPPPPARKGSYVSAFRPVVKDTESIAKLYGSAREAYGAGPARGPGPGAGSGGYVSPDFLSEGSSSYNSASPDVDTADEPEVDVESNRFPDDEDAQEETEPSAPSAGGGPDGEQPTGPPSATSSGADGPANSPDGGSPRPRRRLGPPPAGRPAFGDLAAEDLVRRPERSPPSGGGGYELREPCGPLGGPAPAKVFAPERDEHVKSAAVALGPAASYVCTPEAHEPDKEDNHSPADDLETRKSYPDQRSISQPSPANTDRGEDGLTLDVTGTHLVEKDIENLAREELQKLLLEQMELRKKLEREFQSLKDNFQDQMKRELAYREEMVQQLQIVRDTLCNELDQERKARYAIQQKLKEAHDALHHFSCKMLTPRHCTGNCSFKPPLLP

SEQ ID NO: 92 - protein, 709 aa, Homo sapiens
MAETLEFNDVYQEVKGSMNDGRLRLSRQGIIFKNSKTGKVDNIQAGELTEGIWRRVALGHGLKLLTKNGHVYKYDGFRESEFEKLSDFFKTHYRLELMEKDLCVKGWNWGTVKFGGQLLSFDIGDQPVFEIPLSNVSQCTTGKNEVTLEFHQNDDAEVSLMEVRFYVPPTQEDGVDPVEAFAQNVLSKADVIQATGDAICIFRELQCLTPRGRYDIRIYPTFLHLHGKTFDYKIPYTTVLRLFLLPHKDQRQMFFVISLDPPIKQGQTRYHFLILLFSKDEDISLTLNMNEEEVEKRFEGRLTKNMSGSLYEMVSRVMKALVNRKITVPGNFQGHSGAQCITCSYKASSGLLYPLERGFIYVHKPPVHIRFDEISFVNFARGTTTTRSFDFEIETKQGTQYTFSSIEREEYGKLFDFVNAKKLNIKNRGLKEGMNPSYDEYADSDEDQHDAYLERMKEEGKIREENANDSSDDSGEETDESFNPGEEEEDVAEEFDSNASASSSSNEGDSDRDEKKRKQLKKAKMAKDRKSRKKPVEVKKGKDPNAPKRPMSAYMLWLNASREKIKSDHPGISITDLSKKAGEIWKGMSKEKKEEWDRKAEDARRDYEKAMKEYEGGRGESSKRDKSKKKKKVKVKMEKKSTPSRGSSSKSSSRQLSESFKSKEFVSSDESSSGENKSKKKRRRSEDSEEEELASTPPSSEDSASGSDE

SEQ ID NO: 93 - protein, 1712 aa, Homo sapiens
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 94 - protein, 152 aa, Homo sapiens
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 95 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 96 - protein, 472 aa, Homo sapiens
MAGGEAGVTLGQPHLSRQDLTTLDVTKLTPLSHEVISRQATINIGTIGHVAHGKSTVVKAISGVHTVRFKNELERNITIKLGYANAKIYKLDDPSCPRPECYRSCGSSTPDEFPTDIPGTKGNFKLVRHVSFVDCPGHDILMATMLNGAAVMDAALLLIAGNESCPQPQTSEHLAAIEIMKLKHILILQNKIDLVKESQAKEQYEQILAFVQGTVAEGAPIIPISAQLKYNIEVVCEYIVKKIPVPPRDFTSEPRLIVIRSFDVNKPGCEVDDLKGGVAGGSILKGVLKVGQEIEVRPGIVSKDSEGKLMCKPIFSKIVSLFAEHNDLQYAAPGGLIGVGTKIDPTLCRADRMVGQVLGAVGALPEIFTELEISYFLLRRLLGVRTEGDKKAAKVQKLSKNEVLMVNIGSLSTGGRVSAVKADLGKIVLTNPVCTEVGEKIALSRRVEKHWRLIGWGQIRRGVTIKPTVDDD

SEQ ID NO: 97 - protein, 572 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 98 - protein, 2115 aa, Homo sapiens
MTLHATRGAALLSWVNSLHVADPVEAVLQLQDCSIFIKIIDRIHGTEEGQQILKQPVSERLDFVCSFLQKNRKHPSSPECLVSAQKVLEGSELELAKMTMLLLYHSTMSSKSPRDWEQFEYKIQAELAVILKFVLDHEDGLNLNEDLENFLQKAPVPSTCSSTFPEELSPPSHQAKREIRFLELQKVASSSSGNNFLSGSPASPMGDILQTPQFQMRRLKKQLADERSNRDELELELAENRKLLTEKDAQIAMMQQRIDRLALLNEKQAASPLEPKELEELRDKNESLTMRLHETLKQCQDLKTEKSQMDRKINQLSEENGDLSFKLREFASHLQQLQDALNELTEEHSKATQEWLEKQAQLEKELSAALQDKKCLEEKNEILQGKLSQLEEHLSQLQDNPPQEKGEVLGDVLQLETLKQEAATLAANNTQLQARVEMLETERGQQEAKLLAERGHFEEEKQQLSSLITDLQSSISNLSQAKEELEQASQAHGARLTAQVASLTSELTTLNATIQQQDQELAGLKQQAKEKQAQLAQTLQQQEQASQGLRHQVEQLSSSLKQKEQQLKEVAEKQEATRQDHAQQLATAAEEREASLRERDAALKQLEALEKEKAAKLEILQQQLQVANEARDSAQTSVTQAQREKAELSRKVEELQACVETARQEQHEAQAQVAELELQLRSEQQKATEKERVAQEKDQLQEQLQALKESLKVTKGSLEEEKRRAADALEEQQRCISELKAETRSLVEQHKRERKELEEERAGRKGLEARLQQLGEAHQAETEVLRRELAEAMAAQHTAESECEQLVKEVAAWRERYEDSQQEEAQYGAMFQEQLMTLKEECEKARQELQEAKEKVAGIESHSELQISRQQNELAELHANLARALQQVQEKEVRAQKLADDLSTLQEKMAATSKEVARLETLVRKAGEQQETASRELVKEPARAGDRQPEWLEEQQGRQFCSTQAALQAMEREAEQMGNELERLRAALMESQGQQQEERGQQEREVARLTQERGRAQADLALEKAARAELEMRLQNALNEQRVEFATLQEALAHALTEKEGKDQELAKLRGLEAAQIKELEELRQTVKQLKEQLAKKEKEHASGSGAQSEAAGRTEPTGPKLEALRAEVSKLEQQCQKQQEQADSLERSLEAERASRAERDSALETLQGQLEEKAQELGHSQSALASAQRELAAFRTKVQDHSKAEDEWKAQVARGRQEAERKNSLISSLEEEVSILNRQVLEKEGESKELKRLVMAESEKSQKLEERLRLLQAETASNSARAAERSSALREEVQSLREEAEKQRVASENLRQELTSQAERAEELGQELKAWQEKFFQKEQALSTLQLEHTSTQALVSELLPAKHLCQQLQAEQAAAEKRHREELEQSKQAAGGLRAELLRAQRELGELIPLRQKVAEQERTAQQLRAEKASYAEQLSMLKKAHGLLAEENRGLGERANLGRQFLEVELDQAREKYVQELAAVRADAETRLAEVQREAQSTARELEVMTAKYEGAKVKVLEERQRFQEERQKLTAQVEQLEVFQREQTKQVEELSKKLADSDQASKVQQQKLKAVQAQGGESQQEAQRLQAQLNELQAQLSQKEQAAEHYKLQMEKAKTHYDAKKQQNQELQEQLRSLEQLQKENKELRAEAERLGHELQQAGLKTKEAEQTCRHLTAQVRSLEAQVAHADQQLRDLGKFQVATDALKSREPQAKPQLDLSIDSLDLSCEEGTPLSITSKLPRTQPDGTSVPGEPASPISQRLPPKVESLESLYFTPIPARSQAPLESSLDSLGDVFLDSGRKTRSARRRTTQIINITMTKKLDVEEPDSANSSFYSTRSAPASQASLRATSSTQSLARLGSPDYGNSALLSLPGYRPTTRSSARRSQAGVSSGAPPGRNSFYMGTCQDEPEQLDDWNRIAELQQRNRVCPPHLKTCYPLESRPSLSLGTITDEEMKTGDPQETLRRASMQPIQIAEGTGITTRQQRKRVSLEPHQGPGTPESKKATSCFPRPMTPRDRHEGRKQSTTEAQKKAAPASTKQADRRQSMAFSILNTPKKLGNSLLRRGASKKALSKASPNTRSGTRRSPRIATTTASAATAAAIGATPRAKGKAKH

SEQ ID NO: 99 - protein, 1357 aa, Homo sapiens
MEFYESAYFIVLIPSIVITVIFLFFWLFMKETLYDEVLAKQKREQKLIPTKTDKKKAEKKKNKKKEIQNGNLHESDSESVPRDFKLSDALAVEDDQVAPVPLNVVETSSSVRERKKKEKKQKPVLEEQVIKESDASKIPGKKVEPVPVTKQPTPPSEAAASKKKPGQKKSKNGSDDQDKKVETLMVPSKRQEALPLHQETKQESGSGKKKASSKKQKTENVFVDEPLIHATTYIPLMDNADSSPVVDKREVIDLLKPDQVEGIQKSGTKKLKTETDKENAEVKFKDFLLSLKTMMFSEDEALCVVDLLKEKSGVIQDALKKSSKGELTTLIHQLQEKDKLLAAVKEDAAATKDRCKQLTQEMMTEKERSNVVITRMKDRIGTLEKEHNVFQNKIHVSYQETQQMQMKFQQVREQMEAEIAHLKQENGILRDAVSNTTNQLESKQSAELNKLRQDYARLVNELTEKTGKLQQEEVQKKNAEQAATQLKVQLQEAERRWEEVQSYIRKRTAEHEAAQQDLQSKFVAKENEVQSLHSKLTDTLVSKQQLEQRLMQLMESEQKRVNKEESLQMQVQDILEQNEALKAQIQQFHSQIAAQTSASVLAEELHKVIAEKDKQIKQTEDSLASERDRLTSKEEELKDIQNMNFLLKAEVQKLQALANEQAAAAHELEKMQQSVYVKDDKIRLLEEQLQHEISNKMEEFKILNDQNKALKSEVQKLQTLVSEQPNKDVVEQMEKCIQEKDEKLKTVEELLETGLIQVATKEEELNAIRTENSSLTKEVQDLKAKQNDQVSFASLVEELKKVIHEKDGKIKSVEELLEAELLKVANKEKTVQDLKQEIKALKEEIGNVQLEKAQQLSITSKVQELQNLLKGKEEQMNTMKAVLEEKEKDLANTGKWLQDLQEENESLKAHVQEVAQHNLKEASSASQFEELEIVLKEKENELKRLEAMLKERESDLSSKTQLLQDVQDENKLFKSQIEQLKQQNYQQASSFPPHEELLKVISEREKEISGLWNELDSLKDAVEHQRKKNNDLREKNWEAMEALASTEKMLQDKVNKTSKERQQQVEAVELEAKEVLKKLFPKVSVPSNLSYGEWLHGFEKKAKECMAGTSGSEEVKVLEHKLKEADEMHTLLQLECEKYKSVLAETEGILQKLQRSVEQEENKWKVKVDESHKTIKQMQSSFTSSEQELERLRSENKDIENLRREREHLEMELEKAEMERSTYVTEVRELKDLLTELQKKLDDSYSEAVRQNEELNLLKAQLNETLTKLRTEQNERQKVAGDLHKAQQSLELIQSKIVKAAGDTTVIENSDVSPETESSEKETMSVSLNQTVTQLQQLLQAVNQQLTKEKEHYQVLE

SEQ ID NO: 100 - protein, 393 aa, Homo sapiens
MRTLFNLLWLALACSPVHTTLSKSDAKKAASKTLLEKSQFSDKPVQDRGLVVTDLKAESVVLEHRSYCSAKARDRHFAGDVLGYVTPWNSHGYDVTKVFGSKFTQISPVWLQLKRRGREMFEVTGLHDVDQGWMRAVRKHAKGLHIVPRLLFEDWTYDDFRNVLDSEDEIEELSKTVVQVAKNQHFDGFVVEVWNQLLSQKRVGLIHMLTHLAEALHQARLLALLVIPPAITPGTDQLGMFTHKEFEQLAPVLDGFSLMTYDYSTAHQPGPNAPLSWVRACVQVLDPKSKWRSKILLGLNFYGMDYATSKDAREPVVGARYIQTLKDHRPRMVWDSQASEHFFEYKKSRSGRHVVFYPTLKSLQVRLELARELGVGVSIWELGQGLDYFYDLL

SEQ ID NO: 101 - protein, 493 aa, Homo sapiens
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 102 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 103 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 104 - protein, 298 aa, Homo sapiens
MSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 105 - protein, 2541 aa, Homo sapiens
MVALSLKISIGNVVKTMQFEPSTMVYDACRIIRERIPEAPAGPPSDFGLFLSDDDPKKGIWLEAGKALDYYMLRNGDTMEYRKKQRPLKIRMLDGTVKTIMVDDSKTVTDMLMTICARIGITNHDEYSLVRELMEEKKEEITGTLRKDKTLLRDEKKMEKLKQKLHTDDELNWLDHGRTLREQGVEEHETLLLRRKFFYSDQNVDSRDPVQLNLLYVQARDDILNGSHPVSFDKACEFAGFQCQIQFGPHNEQKHKAGFLDLKDFLPKEYVKQKGERKIFQAHKNCGQMSEIEAKVRYVKLARSLKTYGVSFFLVKEKMKGKNKLVPRLLGITKECVMRVDEKTKEVIQEWNLTNIKRWAASPKSFTLDFGDYQDGYYSVQTTEGEQIAQLIAGYIDIILKKKKSKDHFGLEGDEESTMLEDSVSPKKSTVLQQQYNRVGKVEHGSVALPAIMRSGASGPENFQVGSMPPAQQQITSGQMHRGHMPPLTSAQQALTGTINSSMQAVQAAQATLDDFDTLPPLGQDAASKAWRKNKMDESKHEIHSQVDAITAGTASVVNLTAGDPAETDYTAVGCAVTTISSNLTEMSRGVKLLAALLEDEGGSGRPLLQAAKGLAGAVSELLRSAQPASAEPRQNLLQAAGNVGQASGELLQQIGESDTDPHFQDALMQLAKAVASAAAALVLKAKSVAQRTEDSGLQTQVIAAATQCALSTSQLVACTKVVAPTISSPVCQEQLVEAGRLVAKAVEGCVSASQAATEDGQLLRGVGAAATAVTQALNELLQHVKAHATGAGPAGRYDQATDTILTVTENIFSSMGDAGEMVRQARILAQATSDLVNAIKADAEGESDLENSRKLLSAAKILADATAKMVEAAKGAAAHPDSEEQQQRLREAAEGLRMATNAAAQNAIKKKLVQRLEHAAKQAAASATQTIAAAQHAASTPKASAGPQPLLVQSCKAVAEQIPLLVQGVRGSQAQPDSPSAQLALIAASQSFLQPGGKMVAAAKASVPTIQDQASAMQLSQCAKNLGTALAELRTAAQKAQEACGPLEMDSALSVVQNLEKDLQEVKAAARDGKLKPLPGETMEKCTQDLGNSTKAVSSAIAQLLGEVAQGNENYAGIAARDVAGGLRSLAQAARGVAALTSDPAVQAIVLDTASDVLDKASSLIEEAKKAAGHPGDPESQQRLAQVAKAVTQALNRCVSCLPGQRDVDNALRAVGDASKRLLSDSLPPSTGTFQEAQSRLNEAAAGLNQAATELVQASRGTPQDLARASGRFGQDFSTFLEAGVEMAGQAPSQEDRAQVVSNLKGISMSSSKLLLAAKALSTDPAAPNLKSQLAAAARAVTDSINQLITMCTQQAPGQKECDNALRELETVRELLENPVQPINDMSYFGCLDSVMENSKVLGEAMTGISQNAKNGNLPEFGDAISTASKALCGFTEAAAQAAYLVGVSDPNSQAGQQGLVEPTQFARANQAIQMACQSLGEPGCTQAQVLSAATIVAKHTSALCNSCRLASARTTNPTAKRQFVQSAKEVANSTANLVKTIKALDGAFTEENRAQCRAATAPLLEAVDNLSAFASNPEFSSIPAQISPEGRAAMEPIVISAKTMLESAGGLIQTARALAVNPRDPPSWSVLAGHSRTVSDSIKKLITSMRDKAPGQLECETAIAALNSCLRDLDQASLAAVSQQLAPREGISQEALHTQMLTAVQEISHLIEPLANAARAEASQLGHKVSQMAQYFEPLTLAAVGAASKTLSHPQQMALLDQTKTLAESALQLLYTAKEAGGNPKQAAHTQEALEEAVQMMTEAVEDLTTTLNEAASAAGVVGGMVDSITQAINQLDEGPMGEPEGSFVDYQTTMVRTAKAIAVTVQEMVTKSNTSPEELGPLANQLTSDYGRLASEAKPAAVAAENEEIGSHIKHRVQELGHGCAALVTKAGALQCSPSDAYTKKELIECARRVSEKVSHVLAALQAGNRGTQACITAASAVSGIIADLDTTIMFATAGTLNREGTETFADHREGILKTAKVLVEDTKVLVQNAAGSQEKLAQAAQSSVATITRLADVVKLGAASLGAEDPETQVVLINAVKDVAKALGDLISATKAAAGKVGDDPAVWQLKNSAKVMVTNVTSLLKTVKAVEDEATKGTRALEATTEHIRQELAVFCSPEPPAKTSTPEDFIRMTKGITMATAKAVAAGNSCRQEDVIATANLSRRAIADMLRACKEAAYHPEVAPDVRLRALHYGRECANGYLELLDHVLLTLQKPSPELKQQLTGHSKRVAGSVTELIQAAEAMKGTEWVDPEDPTVIAENELLGAAAAIEAAAKKLEQLKPRAKPKEADESLNFEEQILEAAKSIAAATSALVKAASAAQRELVAQGKVGAIPANALDDGQWSQGLISAARMVAAATNNLCEAANAAVQGHASQEKLISSAKQVAASTAQLLVACKVKADQDSEAMKRLQAAGNAVKRASDNLVKAAQKAAAFEEQENETVVVKEKMVGGIAQIIAAQEEMLRKERELEEARKKLAQIRQQQYKFLPSELRDEH

SEQ ID NO: 106 - protein, 470 aa, Homo sapiens
MSQAYSSSQRVSSYRRTFGGAPGFPLGSPLSSPVFPRAGFGSKGSSSSVTSRVYQVSRTSGGAGGLGSLRASRLGTTRTPSSYGAGELLDFSLADAVNQEFLTTRTNEKVELQELNDRFANYIEKVRFLEQQNAALAAEVNRLKGREPTRVAELYEEELRELRRQVEVLTNQRARVDVERDNLLDDLQRLKAKLQEEIQLKEEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEASGYQDNIARLEEEIRHLKDEMARHLREYQDLLNVKMALDVEIATYRKLLEGEESRINLPIQTYSALNFRETSPEQRGSEVHTKKTVMIKTIETRDGEVVSEATQQQHEVL

SEQ ID NO: 107 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 108 - protein, 895 aa, Homo sapiens
MRMSVGLSLLLPLSGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 109 - RNA, 23 nt, Homo sapiens
uguaaacauccuacacucucagc

SEQ ID NO: 110 - DNA, 104 bp, Homo sapiens
attcttctatagttcatgagcatgatgattgggtgttcatgacatgtgtgagatgtgccaccctctgaaccttgttcccacatcggcacattatccatctaacc

SEQ ID NO: 111 - RNA, 20 nt, Homo sapiens
agcccccuggccccaaaccc

SEQ ID NO: 112 - RNA, 22 nt, Homo sapiens
uuguacaugguaggcuuucauu

SEQ ID NO: 113 - RNA, 21 nt, Homo sapiens
ucgaggagcucacagucuagu

SEQ ID NO: 114 - RNA, 22 nt, Homo sapiens
uguaaacauccccgacuggaag

SEQ ID NO: 115 - RNA, 22 nt, Homo sapiens
gaaguuguucgugguggauucg

SEQ ID NO: 116 - RNA, 22 nt, Homo sapiens
cuuucagucggauguuugcagc

SEQ ID NO: 117 - RNA, 22 nt, Homo sapiens
ugagguaguaguuuguacaguu

SEQ ID NO: 118 - RNA, 22 nt, Homo sapiens
auauaauacaaccugcuaagug

SEQ ID NO: 119 - RNA, 22 nt, Homo sapiens
uagcuuaucagacugauguuga

SEQ ID NO: 120 - RNA, 22 nt, Homo sapiens
ugagguaguagauuguauaguu

SEQ ID NO: 121 - RNA, 21 nt, Homo sapiens
gcuggucugcguggugcucgg

SEQ ID NO: 122 - RNA, 22 nt, Homo sapiens
uauguaacacgguccacuaacc

(19) United States
(12) Patent Application Publication
(10) Pub. No.: US 2021/0239716 A1
(43) Pub. Date: Aug. 5, 2021

(54) A METHOD OF DIAGNOSING OR PROGNOSING A NEUROLOGICAL DISORDER

(71) Applicant: University of Ulster, Londonderry (GB)

(72) Inventors: Stephanie DUGUEZ, Derry (GB); William DUDDY, Derry (GB)

(21) Appl. No.: 17/052,074

(22) PCT Filed: May 1, 2019
(86) PCT No.: PCT/EP2019/061169

§ 371 Date: Oct. 30, 2020

(30) Foreign Application Priority Data
May 1, 2018 (GB) 1807178.7

(51) Int. Cl. G01N 33/68; C12Q 1/6883

(52) U.S. Cl. CPC G01N 33/6896; C12Q 1/6883; C12Q 2600/158; G01N 2800/2835; G01N 2800/285; G01N 2800/28

(57) ABSTRACT

The present invention relates to a method of diagnosing or prognosing a neurological disorder in a subject. The method comprises determining the quantitative or qualitative level of one or more biomarkers in a biological sample from the subject; and diagnosing or prognosing the neurological disorder in the subject based on the quantitative or qualitative level of the or each biomarker in the biological sample.

### FIELD OF THE INVENTION

The present invention relates to a method of diagnosing or prognosing a neurological disorder in a subject. The method comprises determining the quantitative or qualitative level of one or more biomarkers in a biological sample from the subject; and diagnosing or prognosing the neurological disorder in the subject based on the quantitative or qualitative level of the or each biomarker in the biological sample.

### BACKGROUND TO THE INVENTION

Amyotrophic lateral sclerosis (ALS), also known as motor neuron disease (MND), is a fatal motor neuron disorder resulting in progressive degeneration and death of upper and lower motor neurons, protein aggregation, severe muscle atrophy and respiratory insufficiency. Median survival is between 2 and 5 years from the onset of symptoms. ALS manifests as either familial ALS (FALS; ˜10% of cases) or sporadic ALS (SALS; −90% of cases). Incidence rate of ALS has been estimated at between 0.3 and 2.6 cases per 100,000 (2.16 per 100,000 person-years in Europe, 2.0 per 100,000 person-years in Ireland).

Studies in animal models and ALS patients show that motor neuron degeneration starts at the neuromuscular junction and that post-synaptic muscle changes may play an active role. Studies have shown that skeletal muscle has a functional secretory activity. The muscle secretome contains exosomes—vesicles that carry out intercellular transport of functional proteins, mRNA, and miRNA. The exosomes may act in an autocrine/paracrine manner on muscle cells or other types of cells and contribute to muscle growth and regeneration, body-wide metabolism, and other functions.

Pathological aggregations of misfolded proteins, such as SOD1, TDP-43, or FUS proteins in affected neurons and also neighbouring glia can be considered as hallmarks of ALS. Several recent studies have shown that these intracellular proteins can be released through vesicle-mediated exocytosis, and then phagocytosed by neighbouring cells allowing a self-perpetuating transmission to adjacent motor neurons. In addition, in familial ALS, mutations of genes involved in autophagy pathways and in multi-vesicular biogenesis (such as ALSIN, VAPB, CHMP2B, and VCP) have been identified. Together, these data strongly suggest a disruption of the endosome and lysosome pathways in sporadic and familial ALS patients—both of these pathways are involved in exosome genesis.

Diagnosis is slow, often requiring a patient to see several specialists over a period of months: the mean time from onset of symptoms to confirmation of diagnosis is 13-18 months. Thus the problem/need is to diagnose ALS more quickly.

### SUMMARY OF THE INVENTION

According to a first aspect of the present invention, there is provided a method of diagnosing or prognosing a neurological disorder in a subject, the method comprising the steps of:
(a) determining the quantitative or qualitative level of one or more biomarkers in a biological sample from the subject; and
(b) diagnosing or prognosing the neurological disorder in the subject based on the quantitative or qualitative level of the or each biomarker in the biological sample;
   wherein the or each biomarker is selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; EEF1 D; TPI1; SSBP1; PSMB8; PSAP; PSMB7; CAPZA1; PRDX1; PODN; MMP1; NUCB2; HMGB1; AHNAK; FNDC1; DENR; PENK; S100A11; RPL29; RPL27; RPL5; SRSF2; SF3B4; SBSN; LYAR; NUDC; CRLF1; TGFB1; CAT; CTSD; DLD; EPB41L2; PSMB9; RPS14; RPL7A; COLEC12; SMARCC2; TAGLN2; CCT4; ASPH; GPS1; CDK13; SH3GL2; HBA1; APEX1; PSMBS; TXN; AXL; RPS8; SET; RPS18; CFL1; DMKN; RPL22; RPL6; FKBP10; PAFAH1B1; S100A13; PSMA6; SERPINE1; CAPRIN1; SEPT7; VTN; ENPP2; NEO1; DKC1; CHI3L1; SKOR1; SSRP1; COL4A2; NME1; CFB; EIF2S3; DPYSL2; NUMA1; KTN1; CHID1; EFEMP1; YWHAZ; LDHB; SDCBP; TLN1; DES; APP; and DAG1.

Preferably, the or each biomarker is selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; and CAPRIN1.

Further preferably, the or each biomarker is selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; CAPRIN1; DES; APP; and DAG1.

Further preferably, the or each biomarker is selected from: PDIA3; CCDC80; HSPA8; SERPINE2; PLEC; DES; APP; and DAG1.

Optionally, the determining step (a) comprises determining the quantitative or qualitative level of two or more biomarkers in the biological sample from the subject.

Further optionally, the determining step (a) comprises determining the quantitative or qualitative level of three, four, five, eight, ten, twenty, twenty five, twenty eight, thirty, forty, fifty, sixty, seventy, eighty, ninety, one hundred, or more biomarkers in the biological sample from the subject.

Optionally, the determining step (a) comprises determining the quantitative or qualitative level of all of the biomarkers in the biological sample from the subject.

Optionally or additionally, the determining step (a) comprises determining the quantitative or qualitative level of each of the biomarkers in the biological sample from the subject.

Optionally, the or each biomarker is a gene. Further optionally, the or each biomarker is a nucleic acid. Still further optionally, the or each biomarker is a deoxyribonucleic acid.

Optionally, the or each biomarker is a translation product of a gene.

Optionally, the or each biomarker is a translation product of a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; EEF1 D; TPI1; SSBP1; PSMB8; PSAP; PSMB7; CAPZA1; PRDX1; PODN; MMP1; NUCB2; HMGB1; AHNAK; FNDC1; DENR; PENK; S100A11; RPL29; RPL27; RPL8; SRSF2; SF3B4; SBSN; LYAR; NUDC; CRLF1; TGFB1; CAT; CTSD; DLD; EPB41L2; PSMB9; RPS14; RPL7A; COLEC12; SMARCC2; TAGLN2; CCT4; ASPH; GPS1; CDK13; SH3GL2; HBA1; APEX1; PSMBS; TXN; AXL; RPS8; SET; RPS18; CFL1; DMKN; RPL22; RPL6; FKBP10; PAFAH1B1; S100A13; PSMA6; SERPINE1; CAPRIN1; SEPT7; VTN; ENPP2; NEO1; DKC1; CHI3L1; SKOR1; SSRP1; COL4A2; NME1; CFB; EIF2S3; DPYSL2; NUMA1; KTN1; CHID1; EFEMP1; YWHAZ; LDHB; SDCBP; TLN1; DES; APP; and DAG1.

Preferably, the or each biomarker is a translation product of a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; and CAPRIN1.

Further preferably, the or each biomarker is a translation product of a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; CAPRIN1; DES; APP; and DAG1.

Further preferably, the or each biomarker is a translation product of a gene selected from: PDIA3; CCDC80; HSPA8; SERPINE2; PLEC; DES; APP; and DAG1.

Optionally, the or each biomarker is a ribonucleic acid.

Optionally, the or each biomarker is a ribonucleic acid having a miRBase Accession Number selected from MIMAT0000244; ENSG00000239126; MIMAT0016865; MIMAT0002813; MIMAT0004697; MIMAT0000245; MIMAT0000737; MIMAT0000088; MIMAT0000414; MIMAT0004955; MIMAT0000076; MIMAT0000067; MIMAT0018084; and MIMAT0004813.

Optionally, the or each biomarker is a ribonucleic acid having a nucleic acid sequence selected from any one or more of SEQ ID Nos 109-122.

Optionally, the or each biomarker is a protein. Further optionally, the or each biomarker is a peptide. Still further optionally, the or each biomarker is a polypeptide.

Optionally, the or each biomarker is a protein encoded by a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; EEF1 D; TPI1; SSBP1; PSMB8; PSAP; PSMB7; CAPZA1; PRDX1; PODN; MMP1; NUCB2; HMGB1; AHNAK; FNDC1; DENR; PENK; S100A11; RPL29; RPL27; RPL8; SRSF2; SF3B4; SBSN; LYAR; NUDC; CRLF1; TGFB1; CAT; CTSD; DLD; EPB41L2; PSMB9; RPS14; RPL7A; COLEC12; SMARCC2; TAGLN2; CCT4; ASPH; GPS1; CDK13; SH3GL2; HBA1; APEX1; PSMBS; TXN; AXL; RPS8; SET; RPS18; CFL1; DMKN; RPL22; RPL6; FKBP10; PAFAH1B1; S100A13; PSMA6; SERPINE1; CAPRIN1; SEPT7; VTN; ENPP2; NEO1; DKC1; CHI3L1; SKOR1; SSRP1; COL4A2; NME1; CFB; EIF2S3; DPYSL2; NUMA1; KTN1; CHID1; EFEMP1; YWHAZ; LDHB; SDCBP; TLN1; DES; APP; and DAG1.

Preferably, the or each biomarker is a protein encoded by a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; and CAPRIN1.

Further preferably, the or each biomarker is a protein encoded by a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; CAPRIN1; DES; APP; and DAG1.

Further preferably, the or each biomarker is a protein encoded by a gene selected from: PDIA3; CCDC80; HSPA8; SERPINE2; PLEC; DES; APP; and DAG1.

Optionally, the or each biomarker is a protein having a UniProtKB/Swiss-Prot Accession Number selected from: P30101; Q76M96; P11142; P07093; Q15149; P11021; P19338; P09493; P24821; P17936; P20908; Q9Y240; P46821; Q13765; P25787; P46777; P60660; P07858; P10809; Q6NZI2; Q02818; P13667; P07237; 043707; P29692; P60174; Q04837; P28062; P07602; Q99436; P52907; Q06830; Q7Z5L7; P03956; P80303; P09429; Q09666; Q4ZHG4; 043583; P01210; P31949; P47914; P61353; P62917; Q01130; Q15427; Q6UWP8; Q9NX58; Q9Y266; 075462; P01137; P04040; P07339; P09622; 043491; P28065; P62263; P62424; Q5KU26; Q8TAQ2; P37802; P50991; Q12797; Q13098; Q14004; Q99962; P69905; P27695; P28074; P10599; P30530; P62241; Q01105; P62269; P23528; Q6E0U4; P35268; Q02878; Q96AY3; P43034; Q99584; P60900; P05121; Q14444; Q16181; P04004; Q13822; Q92859; 060832; P36222; P84550; Q08945; P08572; P15531; P00751; P41091; Q16555; Q14980; Q86UP2; Q9BWS9; Q12805; P63104; P07195; 000560; Q9Y490; P17661; P05067; and Q14118.

Preferably, the or each biomarker is a protein having a UniProtKB/Swiss-Prot Accession Number selected from: P30101; Q76M96; P11142; P07093; Q15149; P11021; P19338; P09493; P24821; P17936; P20908; Q9Y240; P46821; Q13765; P25787; P46777; P60660; P07858; P10809; Q6NZI2; Q02818; P13667; P07237; 043707; and Q14444.

Further preferably, the or each biomarker is a protein having a UniProtKB/Swiss-Prot Accession Number selected from: P30101; Q76M96; P11142; P07093; Q15149; P11021; P19338; P09493; P24821; P17936; P20908; Q9Y240; P46821; Q13765; P25787; P46777; P60660; P07858; P10809; Q6NZI2; Q02818; P13667; P07237; 043707; Q14444; P17661; P05067; and Q14118.

Further preferably, the or each biomarker is a protein having a UniProtKB/Swiss-Prot Accession Number selected from: P30101; Q76M96; P11142; P07093; Q15149; P17661; P05067; and Q14118.

Optionally, the or each biomarker is a protein having an amino acid sequence selected from any one or more of SEQ ID Nos 1-108.

Optionally, the neurological disorder is a neurodegenerative disorder.

Optionally, the neurodegenerative disorder is selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally, the diagnosing or prognosing step (b) comprises comparing the quantitative or qualitative level of the or each biomarker in the biological sample from the subject with the quantitative or qualitative level of the or each respective biomarker in a normal sample.

Optionally, the normal sample is a biological sample from a subject not suffering from a neurological disorder, optionally a neurodegenerative disorder, further optionally a neurodegenerative disorder selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally, a quantitative or qualitative level of the or each biomarker in the biological sample from the subject greater than the quantitative or qualitative level of the or each respective biomarker in a normal sample is indicative of the quantitative or qualitative level of the neurological disorder, optionally the neurodegenerative disorder, further optionally the neurodegenerative disorder selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally, a quantitative or qualitative level of the or each biomarker in the biological sample from the subject greater than the quantitative or qualitative level of the or each respective biomarker in a normal sample is indicative of the quantitative or qualitative presence of the neurological disorder, optionally the neurodegenerative disorder, further optionally the neurodegenerative disorder selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally, the determining step (a) comprises determining the quantitative or qualitative level of one or more subsets of one or more biomarkers in the biological sample from the subject.

Optionally, the determining step (a) comprises determining the quantitative or qualitative level of two or more subsets of one or more biomarkers in the biological sample from the subject.

Optionally, the determining step (a) comprises determining the quantitative or qualitative level of three, four, five, six, seven, eight, nine, ten, eleven, twelve, thirteen, or fourteen subsets of one or more biomarkers in the biological sample from the subject.

Optionally, the determining step (a) comprises determining the quantitative or qualitative level of one or more of a first, second, third, fourth, fifth, sixth, seventh, eighth, ninth, tenth, eleventh, twelfth, thirteenth, or fourteenth subset of one or more biomarkers in the biological sample from the subject.

Optionally, the first subset comprises one or more biomarkers selected from: PDIA3; and CCDC80.

Optionally, the second subset comprises HSPA8.

Optionally, the third subset comprises one or more biomarkers selected from: SERPINE2; and PLEC.

Optionally, the fourth subset comprises one or more biomarkers selected from: HSPA5; and NCL.

Optionally, the fifth subset comprises one or more biomarkers selected from: TPM1; and TNC.

Optionally, the sixth subset comprises IGFBP3.

Optionally, the seventh subset comprises one or more biomarkers selected from: COL5A1; CLEC11A; MAP1B; NACA; and PSMA2.

Optionally, the eighth subset comprises one or more biomarkers selected from: RPL5; MYL6; CTSB; HSPD1; PTRF; NUCB1; PDIA4; P4HB; and ACTN4.

Optionally, the ninth subset comprises one or more biomarkers selected from: EEF1 D; TPI1; SSBP1; PSMB8; PSAP; PSMB7; CAPZA1; PRDX1; PODN; MMP1; NUCB2; HMGB1; AHNAK; and FNDC1.

Optionally, the tenth subset comprises one or more biomarkers selected from: DENR; PENK; S100A11; RPL29; RPL27; RPL8; SRSF2; SF3B4; SBSN; LYAR; NUDC; CRLF1; TGFB1; CAT; CTSD; DLD; EPB41L2; PSMB9; RPS14; RPL7A; COLEC12; SMARCC2; TAGLN2; CCT4; ASPH; GPS1; CDK13; SH3GL2; HBA1; APEX1; PSMBS; TXN; AXL; RPS8; SET; RPS18; CFL1; DMKN; RPL22; RPL6; FKBP10; PAFAH1B1; S100A13; PSMA6; SERPINE1; CAPRIN1; SEPT7; VTN; ENPP2; NEO1; DKC1; CHI3L1; SKOR1; SSRP1; COL4A2; NME1; CFB; EIF2S3; DPYSL2; NUMA1; KTN1; CHID1; EFEMP1; YWHAZ; LDHB; SDCBP; and TLN1.

Optionally, the eleventh subset comprises one or more biomarkers selected from: DES; APP; and DAG1.

Optionally, the twelfth subset comprises one or more biomarkers selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; and CAPRIN1.

Optionally, the thirteenth subset comprises one or more biomarkers selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; CAPRIN1; DES; APP; and DAG1.

Optionally, the fourteenth subset comprises one or more biomarkers selected from: PDIA3; CCDC80; HSPA8; SERPINE2; PLEC; DES; APP; and DAG1.

Optionally, the determining step (a) comprises determining the quantitative or qualitative level of all of the biomarkers in one or more of the first, second, third, fourth, fifth, sixth, seventh, eighth, ninth, tenth, eleventh, twelfth, thirteenth, or fourteenth subsets.

Optionally, the determining step (a) comprises determining the quantitative or qualitative level of each of the biomarkers in one or more of the first, second, third, fourth, fifth, sixth, seventh, eighth, ninth, tenth, eleventh, twelfth, thirteenth, or fourteenth subsets.

Optionally, the diagnosing or prognosing step (b) comprises comparing the quantitative or qualitative level of the or each biomarker in the or each subset in the biological sample from the subject with the quantitative or qualitative level of the or each respective biomarker in a normal sample.

Optionally, the normal sample is a biological sample from a subject not suffering from a neurological disorder, optionally a neurodegenerative disorder, further optionally a neurodegenerative disorder selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally, a quantitative or qualitative level of the or each biomarker in the or each subset in the biological sample from the subject greater than the quantitative or qualitative level of the or each respective biomarker in a normal sample is indicative of the quantitative or qualitative level of the neurological disorder, optionally the neurodegenerative disorder, further optionally the neurodegenerative disorder selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally, a quantitative or qualitative level of the or each biomarker in the or each subset in the biological sample from the subject greater than the quantitative or qualitative level of the or each respective biomarker in a normal sample is indicative of the quantitative or qualitative presence of the neurological disorder, optionally the neurodegenerative disorder, further optionally the neurodegenerative disorder selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

Optionally or additionally, the determining step (a) further comprises the additional step of determining the quantitative or qualitative level of one or more biomarkers in a biological sample from the subject; wherein the or each biomarker is a ribonucleic acid having a miRBase Accession Number selected from MIMAT0000244; ENSG00000239126; MIMAT0016865; MIMAT0002813; MIMAT0004697; MIMAT0000245; MIMAT0000737; MIMAT0000088; MIMAT0000414; MIMAT0004955; MIMAT0000076; MIMAT0000067; MIMAT0018084; and MIMAT0004813.

Optionally, the biological sample is selected from whole blood, serum, plasma, urine, interstitial fluid, peritoneal fluid, cervical swab, tears, saliva, buccal swab, skin, brain tissue, and cerebrospinal fluid.

Further optionally, the biological sample is selected from whole blood, serum, and plasma.

Still further optionally, the biological sample is whole blood.

PDIA3 is synonymous with Erp57.

### BRIEF DESCRIPTION OF THE DRAWINGS

Embodiments of the present invention will now be described with reference to the following non-limiting examples and the accompanying drawings, in which:

FIG. 1 is a schematic illustration of ALS signature in muscle stem cells;

FIG. 2(A) illustrates immunostaining for exosome markers of differentiated muscle stem cells, (B) the secretion of exosomes by muscle cells, and (C) the multi-vesicular bodies of ALS muscle cells contain more exosomes than the controls;

FIG. 3 illustrates representative images of healthy myotubes treated with ALS or Control exosomes, wherein ALS exosomes induce cell stress;

FIG. 4 illustrates boxplots showing expression values of non-coding RNA microarrays (raw: left panel; background corrected: middle panel; background corrected and normalized: right panel);

FIG. 5 illustrates boxplots showing RLE values for non-coding RNA microarrays (average values close to 0 pass quality control);

FIG. 6 illustrates boxplots showing NUSE values for non-coding RNA microarrays (average values<1.05 pass quality control);

FIG. 7 illustrates DAG1 expression level in circulating serum exosomes, using anti-DAG antibody. Values are mean±SD. Serum from 8 ALS patients (1 month after diagnose), 11 healthy subjects and 9 Parkinson patients. ANOVA 1 factor followed by a Tukey post-hoc test was performed. **, significantly different from healthy values, P<0.01;

FIG. 8 illustrates HSPA8 expression level in circulating serum exosomes. Values are mean±SD. Serum from 5 ALS patients (1 month after diagnose), 9 healthy subjects and 7 Parkinson patients. ANOVA 1 factor followed by a Tukey post-hoc test was performed. *, **, significantly different from ALS values, P<0.05 and P<0.01 respectively; and

FIG. 9 illustrates levels of each biomarker in exosomes isolated from the blood serum of ALS patients, normalized against the quantity of total blood serum exosome (CD63), and plotted against the rate of progression of the pathology (delta ALSFRS-R).

### EXAMPLES

### Materials & Methods

### Participants and Ethical Approval

An open biopsy was performed in deltoid muscle on 18 ALS patients who attended the Motor Neuron Diseases center (Pitié Salpétrière, Paris). Twenty-one deltoid muscle biopsies from healthy age and gender-matched subjects were obtained from the BTR (Bank of Tissues for Research, a partner in the EU network EuroBioBank) in accordance with European recommendations and French legislation. The protocol (NCT01984957) was approved by the local ethical committee and all subjects completed informed consent in accordance with institutional guidelines.

### Muscle Stem Cell Extraction and Culture

Muscle biopsies were dissociated mechanically and plated in proliferation medium as previously described (Mamchaoui et al, Skeletal muscle, 2011, PMID: 22040608). CD56 magnetic bead sorting system (MACS, Miltenyl Biotech) was used to sort the myogenic cell population in accordance with the manufacturer's instructions. The myogenicity of the cell cultures was checked before and after sorting the cells by immuno-labelling using anti-desmin antibodies, and the cells were differentiated for 3 days in Dulbecco's Modified Eagle's medium (DMEM). A minimum of 80% of the cell population were positive for desmin.

Exosomes Extraction from the Culture Medium

Briefly, 10 million muscle stem cells (ALS and healthy subject, n=3 per group) were differentiated in DMEM for 3 days. After 3 days, the conditioned media were harvested, centrifuged at 300×g for 5 min at room temperature (RT) and then at 4000 g for 20 min at 4° C. in order to remove any dead cells and cell debris. The supernatants were filtered through 0.22 μm filter to remove the microparticles. The samples used for protein and miRNA extraction, and stored at −80° C.

Protein Extraction from Exosomes

A volume of cell culture medium was mixed to 0.5 volumes of Total Exosome Isolation Reagent from cell culture media (lifeTechnologies®) and incubated overnight at 4° C. The samples were then centrifugated at 10,000×g for 1 hr at 4° C. The exosome pellets were re-suspended in 25 μl 8M Urea, 50 mM ammonium bicarbonate, pH 8.5, and reduced with dithiothreitol (DTT) for 1 h at 4° C. Protein concentrations were then quantified using Pierce BCA Protein Assay kit (ThermoFisher®). Exosomal proteins were kept at −80° C.

### Proteome Profile Determined by Mass Spectrometry

Approximately 20 μg of exosome protein were trypsin digested using a SmartDigest column (Thermo) for 2 hours at 70° C. and 1400 rpm in accordance with the manufacturer's instructions. Peptides were then fractionated into 8 fractions using a high pH reverse phase spin column (Thermo) in accordance with the manufacturer's instructions. Fractioned peptides were vacuum dried, re-suspended and analyzed by data-dependent mass spectrometry on a Q Exactive HF (Thermo) with the following parameters: Positive Polarity, m/z 400-2000 MS Resolution 70,000, AGC 3e6, 100 ms IT, MS/MS Resolution 17,500, AGC 5e5, 50 ms IT, Isolation width 3 m/z, and NCE 30, cycle count 15.

### Database Search and Quantification

Resulting mass spectral files were searched for protein identification using ProteomeDiscoverer (Thermo) against the Uniprot human database for semi tryptic peptides and filtered based on a false discovery rate of <1%.

### Proteome Profile Analysis

Proteins were selected as candidates if the range of observed abundance of the 3 ALS replicates did not overlap with the range of the 3 Healthy control replicates. Candidates were ranked based on the difference in the median value of the 3 ALS replicates from that of the Healthy replicates.

miRNA Extraction from Exosomes

A volume of cell culture medium was mixed to 0.5 volumes of Total Exosome Isolation Reagent from cell culture media (lifeTechnologies®) and incubated overnight at 4° C. The samples were then centrifugated at 10,000×g for 1 hr at 4° C. The exosome pellets were re-suspended with 1 ml of trizol. After adding 0.2 mL of chloroform, the samples were incubated for 3 minutes at room temperature, then centrifugated for 15 minutes at 12,000×g at 4° C. The aqueous upper phase was then mixed with ⅓ volume of 100% ethanol and mixed thoroughly. The samples were then placed on place a filter cartridge into a collection tube of total RNA and protein isolation kit from Invitrogen™ (LifeTechnologies™). The miRNA extraction was performed following the manufacturer's instruction. RNA quality was determined using Eukaryote Total RNA Nano 2.6 (Agilent) and 2100 bioanalyzer. RNA quantity was determined using nanodrop, each in accordance with the manufacturer's instructions.

miRNA Profile and Analysis

Total RNA (150 ng) was poly(A) tailed and biotin labelled using a FlashTag Biotin HSR RNA labelling kit and hybridized to Affymetrix GeneChip miRNA 4.0 arrays for 16 hours (48° C.), following the manufacturer's instructions (ThermoFisher Scientific, Waltham, Mass.). Hybridization cocktails were then removed and the arrays washed and stained on a Fluidics Station 450 with the fluidics script for miRNA 4.0 arrays. Finally, arrays were scanned on a Affymetrix GCS3000 7G scanner and initial quality control data evaluated using Affymetrix Expression Console software (both from ThermoFisher Scientific).

miRNome Profile Analysis

Further quality control of raw data was carried out using the R/Bioconductor packages oligo and pd.mirna.4.0. These packages were used to evaluate each sample's distribution of expression values, relative log expression values (RLE) values, and normalized unscaled standard errors (NUSE), all of which were consistent with good quality. Expression matrices were then normalized by quantile normalization and detection above background using Affymetrix Expression Console v 1.4.1.46. Differentially expressed miRNA (ALS v Healthy) were then identified as those having absolute fold-change>2 and one-way, unpaired ANOVA p-value<0.05.

Serum Samples from Patients

Blood samples were collected using silica vacutainers, then gently inverted 8-10 times. After 2h of incubation on ice to allow clotting, samples were centrifugated at 1,500 g for 15 mn. Aliquots of 100 μl of serum were transferred to cryovials and stored at −80° C. Eight ALS patients, 11 healthy subjects and 9 Parkinson patients aged and gender matched, were recruited (NCT01302600 and NCT02305147).

Exosome Extraction from Serum

The exosomes were precipitated by adding 6 μl Total Exosome Isolation Reagent, vortexing and incubating for 30 mn on ice. Samples were then centrifugated for 10 mn at 10,000 g at RT. The pellets were resuspended in 13.5 μl of Urea 4M/RIPA and kept on ice. In parallel, 6.3111 of Total Exosome Isolation Reagent was added to the remaining supernatants, vortexed and incubated for 30 mn on ice. These second mix were then centrifugated for 10 mn at 10,000 g at RT. The second exosome pellets were resuspended in 13.5 μl of Urea 4M/RIPA. The two exosome protein extracts were then mixed together and used for dot-blot analysis. The exosome protein extracts were stored at −80° C.

### Sample Preparation for the Dot Blot

To 5111 of exosome protein extract was added 1×NuPAGE (Invitrogen™). These non-reduced samples were used for CD63 analysis. To the remaining 22 μl of the exosome protein extract was added 1×NuPAGE (Invitrogen™) supplemented with 1× reducing reagent (Invitrogen™). All samples were then heated at 70° C. for 10 mn.

### Dot Blot Analysis

1 μl of reduced or non-reduced sample was loaded on the methanol pre-activated 0.2 μm PVDF (Immobilon®, Sigma-Aldrich®). After drying for 5 mn at RT, the membranes were re-activated quickly in methanol and staining with Ponceau S (Sigma-Aldrich®) to control the loading. The membranes were then blocked overnight at 4° C. in TBS-0.01% tween (TBS-T) supplemented with 5% milk. The primary antibodies for CD63 (TS63, Invitrogen™), for beta-dystroglycan (MANDAG2, 7D11, DSHB) and for HSPA8 (Millipore®) were extemporaneously coupled with biotin using Zenon™ BiotinXX mouse IgG1 labelling kit following the manufacturer instructions. Briefly, to 1 μg of antibody was added first 5 μl of complex, then 5 μl of blocking buffer. Anti-CD63, anti-betadystroglycan, and anti-HSPA8 were diluted in 1/1000, 1/133 and 1/500 respectively in TBS-T-5% milk.

The primary antibodies for Serpin E2 (TS63, Invitrogen™), for ERP57 (PDIA3, Merck-Millipore®), Desmin (Y66, Invitrogen™), for Amyloid Precursor Protein (abcam), CCDC80 (Invitrogen™), and Plectin (Invitrogen™) were extemporaneously coupled with biotin using Zenon™ BiotinXX rabbit IgG labelling kit following the manufacturer instructions. Briefly, to 1 μg of antibody was added first 5 μl of complex, then 5 μl of blocking buffer. Anti-Serpin E2 anti-ERP57, anti-Desmin, anti-Amyloid Precursor Protein, anti-CCDC80, and anti-Plectin were diluted in 1/1000 in TBS-T-5% milk.

Membranes were incubated with diluted primary biotin-coupled antibodies for 45 mn at RT. Membranes were then rinsed 3 times in TBS-T for 5 mn. Membranes were then incubated for 45 min at RT with streptavidin-HRP (Invitrogen™) diluted at 1/250 in TBS-T-5% milk. Membranes were then rinsed 3 times in TBS-T for 5 mn. Membranes were revealed using SuperSignal™ West Pico PLUS Chemiluminescent Substrate (Thermo Scientific™). Images were acquired every 15s for 15 mn using UVP/ChemiDoc-It®2 Imager. Images were then analysed using FIJI software.

### Statistical Analysis

Values are mean±SD. An ANOVA 1 Factor followed by a Tukey posthoc-test was performed to assess the differences in biomarker levels between ALS, healthy and Parkinson subjects. The correlation between the biomarker levels and the progression of ALSFRS was tested using a Pearson correlation analysis. Cut-off for significant differences was set at P<0.05.

### Example 1—Identification of ALS Signature in Muscle Stem Cells

Muscle stem cells were extracted from muscle biopsies of patients and healthy subjects (all male, 30-67 years old, n=5/6 per group), purified (>80% myogenic cells) and differentiated for 3 days into myotubes, wherein the samples were analyzed using GeneChip Human Exon 1.0ST array (Affymetrix, Inc., Cleveland, Ohio); and (A) illustrates a PCA plot showing clear separation of ALS patients on the 2nd component (y-axis), ALS: blue, SBMA: pink, SMA-III/IV: red, Control: green; (B) illustrates, of the 30 genes having expression signatures most strongly specific to ALS, many have been observed at exosomally-relevant subcellular localizations, according to analysis by the CellWhere tool.

### Example 2—ALS Muscle Stem Cells Accumulate and Secrete More Exosomes than Controls

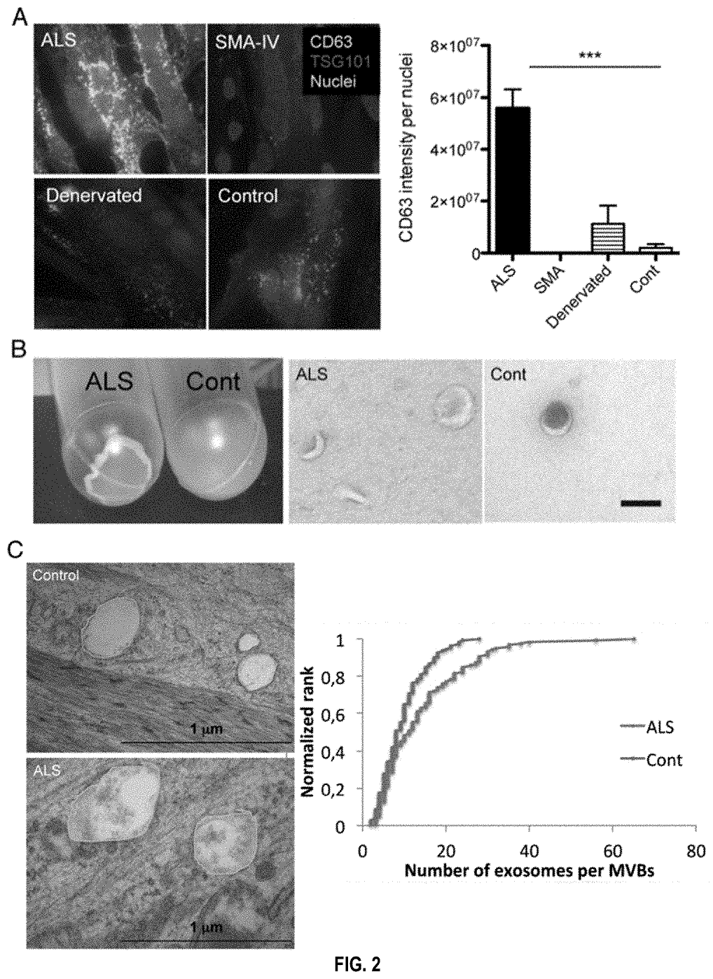

Referring to FIG. 2(A), there is shown immunostaining for exosome markers of differentiated muscle stem cells. Left panel: Representative images of cultured muscle stem cells from ALS and SMA-III/IV patients, and from healthy control or denervated subjects labelled for exosomal markers (TSG101, CD63). Right Panel: quantification of CD63 fluorescence signal normalized per myonucleus. (n=8, 4, 2, 5 cell culture of different ALS, SMA-III/IV, polyneuritis and control subjects). ***, P<0.001.

FIG. 2(B) illustrates exosomes are oversecreted by ALS muscle cells. Left panel: Representative picture of the exosome pellets from culture medium. The exosomes were extracted from the culture medium of 800,000 cells from ALS and control subjects. The experiment has been repeated 8 times on different cultures of muscle cells from ALS patients and control subjects. Right panel: electron microscopy confirming the presence of exosomes (cup-shape vesicles) in the pellets. The exosomes were more numerous in ALS pellets. Of note, the immunostaining performed on these vesicles confirmed that they are exosomes as they are positive for CD63 and CD82.

FIG. 2(C) shows the multi-vesicular bodies of ALS muscle cells contain more exosomes than the controls. Left panel: Representative electron microscopy images showing an accumulation of exosomes in multi-vesicular bodies (MVBs, highlighted in green and orange) of ALS muscle cells compared to controls. Scale bar=1 μm. Right panel: quantification of the number of exosome-like vesicles in the MVBs. Two sample Kolmogorov-Smirnov test confirmed the visual impression that the MVBs of ALS muscle cells contain a greater number of exosome-like vesicles compare to the control (P<0.05).

### Example 3—Exosomes from ALS Muscle Cells are Toxic to Healthy Myotubes and Motor Neurons

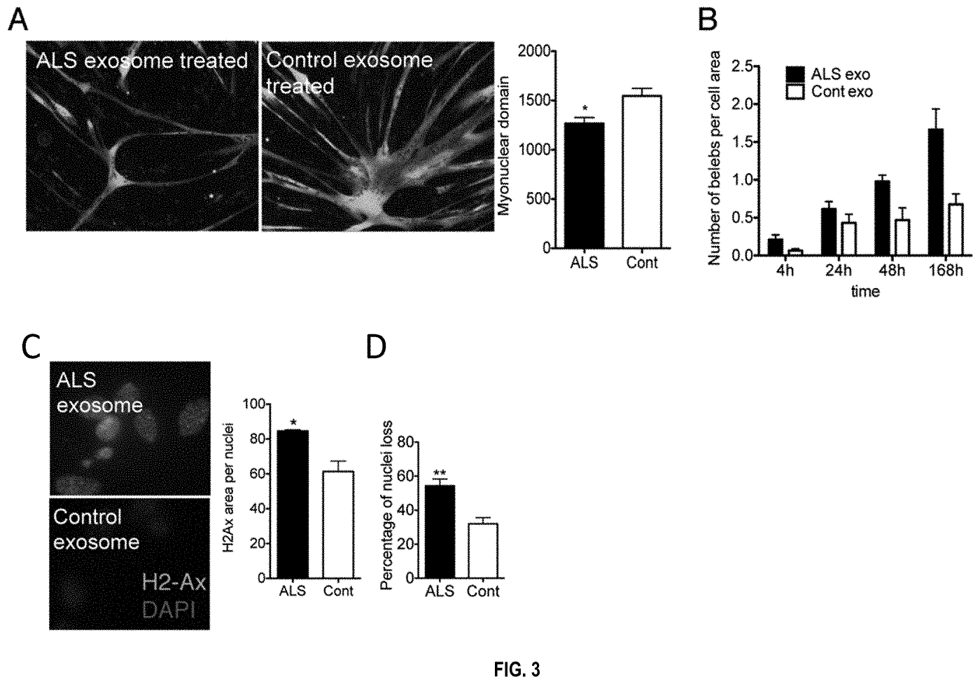

Referring to FIG. 3(A), it is shown that ALS exosomes induce muscle cell atrophy. Left panel: Representative images of healthy myotubes treated with ALS or Control exosomes. Myotubes are stained with myosin heavy chain (green). Right panel: quantification of myonuclear domain size (area of myosin heavy chain staining divided by the number of nuclei). The ALS-exosome treated myotubes have a smaller myonuclear domain (*P<0.05; n=6/group).

FIG. 3(B) illustrates ALS exosomes induce cell stress. Quantification of blebs numbers per healthy myotubes over time treated with ALS or control exosomes (n=6/group). ANOVA 2 factor interaction, time and treatment P<0.001.

FIG. 3(C) illustrates ALS exosomes induce cell death. Left panel: representative immunostaining of H2-Ax immunostaining of healthy myotubes treated with ALS or control exosomes. Middle panel: quantification of H2-Ax signal per nucleus. Right panel: quantification of loss of motor neuron nuclei in culture.

### Example 4—Identification of Specific Exosomal Contents as Biomarkers of ALS

Exosomal contents include various types of biomolecule, of which those potentially involved in signaling roles include proteins and their peptides, and non-coding RNAs. Muscle stem cells (primary myoblasts) were obtained from the Deltoid muscles of ALS patients and healthy controls and cultured to form differentiated myotubes. Exosomes were purified from the culture medium of the 10 million muscle cells following 3 days of culture, using total exosome isolation reagent (Invitrogen™ Life Technologies™). To identify the contents of exosomes, we carried out proteomic and non-coding RNA profiling of purified exosomes.

### Protein Biomarkers

Proteomic mass spectrometry data identified peptides of 105 proteins (Table 1) having levels that were consistently increased in ALS muscle exosomes (n=3) compared to healthy control muscle exosomes (n=3).

Since the proteomic samples were normalized to protein concentration, it was also noted that proteins with consistently high levels in both ALS and Healthy muscle exosomes may also be good biomarkers, as these may still be elevated in ALS blood per volume due to our observation that muscle exosomes are secreted in greater abundance from ALS myotubes than from healthy controls. In this way, a further 3 candidates that had consistently high levels in both ALS and Healthy muscle exosomes, and had other features of mechanistic interest, were identified. Namely, these were DES, APP, and DAG1. APP is the primary component of amyloid plaques in Alzheimer's disease. DES is observed at high levels in all muscle tissue types but is entirely absent from other tissues. DAG1 is a major component of the dystroglycan complex, which is implicated in a number of muscular dystrophies.

TABLE 1
List of 108 proteins of which peptides were identified by mass spectrometry to be
consistently elevated in exosomes of ALS muscle cells compared to healthy controls, or that had
consistently high levels in both ALS and Healthy muscle exosomes, and had other features of
mechanistic relevance to the pathology. The first column gives standard protein IDs used by Uniprot
(http://www.uniprot.org/), and corresponding gene names in column 2. Columns 3-5 and 6-8 show
levels of peptides in ALS samples and healthy control samples, respectively. Column 9 indicates the
difference in median quantities of the protein peptides in ALS compared to control, and column 10
ranks the proteins based on the values in column 9.

| | | | | | | | | Med | |
|---|---|---|---|---|---|---|---|---|---|
| | | | | | | | | ALS-Cont | Rank |
| | | | | | | | | IF no | ALS |
| Uniprot | Gene | SD990- | SD1016- | GO992- | SD1048- | SD1049- | GO849- | overlap | over |
| ID | name | ALS1 | ALS2 | ALS3 | Cont1 | Cont2 | Cont3 | in range | Control |
| P30101 | PDIA3 | 20 | 15 | 35 | 5 | 2 | 3 | 17 | 1 |
| Q76M96 | CCDC80 | 19 | 3 | 57 | 2 | 2 | 0 | 17 | 1 |
| P11142 | HSPA8 | 8 | 11 | 21 | 0 | 0 | 0 | 11 | 3 |
| P07093 | SERPINE2 | 11 | 5 | 16 | 1 | 3 | 0 | 10 | 4 |
| Q15149 | PLEC | 25 | 24 | 52 | 15 | 23 | 3 | 10 | 4 |
| P11021 | HSPA5 | 11 | 9 | 27 | 2 | 4 | 1 | 9 | 6 |
| P19338 | NCL | 9 | 2 | 9 | 1 | 0 | 0 | 9 | 6 |
| P09493 | TPM1 | 8 | 0 | 11 | 0 | 0 | 0 | 8 | 8 |
| P24821 | TNC | 9 | 2 | 35 | 1 | 1 | 0 | 8 | 8 |
| P17936 | IGFBP3 | 9 | 3 | 8 | 3 | 1 | 2 | 6 | 10 |
| P20908 | COL5A1 | 34 | 11 | 12 | 9 | 4 | 8 | 4 | 11 |
| Q9Y240 | CLEC11A | 8 | 5 | 7 | 3 | 4 | 1 | 4 | 11 |
| P46821 | MAP1B | 4 | 0 | 6 | 0 | 0 | 0 | 4 | 11 |
| Q13765 | NACA | 6 | 1 | 4 | 0 | 1 | 0 | 4 | 11 |
| P25787 | PSMA2 | 4 | 2 | 7 | 1 | 0 | 0 | 4 | 11 |
| P46777 | RPL5 | 4 | 3 | 4 | 1 | 1 | 1 | 3 | 16 |
| P60660 | MYL6 | 3 | 3 | 2 | 1 | 0 | 0 | 3 | 16 |
| P07858 | CTSB | 4 | 0 | 3 | 0 | 0 | 0 | 3 | 16 |
| P10809 | HSPD1 | 5 | 0 | 3 | 0 | 0 | 0 | 3 | 16 |
| Q6NZI2 | PTRF | 3 | 0 | 9 | 0 | 0 | 0 | 3 | 16 |
| Q02818 | NUCB1 | 4 | 1 | 12 | 0 | 1 | 1 | 3 | 16 |
| P13667 | PDIA4 | 3 | 3 | 16 | 1 | 0 | 0 | 3 | 16 |
| P07237 | P4HB | 3 | 2 | 29 | 0 | 0 | 0 | 3 | 16 |
| O43707 | ACTN4 | 0 | 3 | 62 | 0 | 0 | 0 | 3 | 16 |
| P29692 | EEF1D | 3 | 1 | 3 | 1 | 1 | 1 | 2 | 25 |
| P60174 | TP11 | 3 | 2 | 6 | 1 | 1 | 1 | 2 | 25 |
| Q04837 | SSBP1 | 2 | 1 | 2 | 1 | 0 | 0 | 2 | 25 |
| P28062 | PSMB8 | 2 | 2 | 1 | 1 | 0 | 0 | 2 | 25 |
| P07602 | PSAP | 2 | 0 | 2 | 0 | 0 | 0 | 2 | 25 |
| Q99436 | PSMB7 | 2 | 1 | 3 | 1 | 0 | 0 | 2 | 25 |
| P52907 | CAPZA1 | 2 | 0 | 3 | 0 | 0 | 0 | 2 | 25 |
| Q06830 | PRDX1 | 2 | 0 | 3 | 0 | 0 | 0 | 2 | 25 |
| Q7Z5L7 | PODN | 2 | 1 | 4 | 0 | 1 | 0 | 2 | 25 |
| P03956 | MMP1 | 2 | 4 | 0 | 0 | 0 | 0 | 2 | 25 |
| P80303 | NUCB2 | 3 | 3 | 7 | 1 | 0 | 2 | 2 | 25 |
| P09429 | HMGB1 | 7 | 2 | 0 | 0 | 0 | 0 | 2 | 25 |
| Q09666 | AHNAK | 2 | 0 | 8 | 0 | 0 | 0 | 2 | 25 |
| Q4ZHG4 | FNDC1 | 2 | 3 | 9 | 1 | 0 | 2 | 2 | 25 |
| O43583 | DENR | 1 | 1 | 0 | 0 | 0 | 0 | 1 | 39 |
| P01210 | PENK | 1 | 1 | 0 | 0 | 0 | 0 | 1 | 39 |
| P31949 | S100A11 | 1 | 1 | 0 | 0 | 0 | 0 | 1 | 39 |
| P47914 | RPL29 | 1 | 1 | 0 | 0 | 0 | 0 | 1 | 39 |
| P61353 | RPL27 | 1 | 1 | 0 | 0 | 0 | 0 | 1 | 39 |
| P62917 | RPL8 | 1 | 1 | 0 | 0 | 0 | 0 | 1 | 39 |
| Q01130 | SRSF2 | 1 | 1 | 0 | 0 | 0 | 0 | 1 | 39 |
| Q15427 | SF3B4 | 1 | 1 | 0 | 0 | 0 | 0 | 1 | 39 |
| Q6UWP8 | SBSN | 1 | 1 | 0 | 0 | 0 | 0 | 1 | 39 |
| Q9NX58 | LYAR | 1 | 1 | 0 | 0 | 0 | 0 | 1 | 39 |
| Q9Y266 | NUDC | 1 | 1 | 0 | 0 | 0 | 0 | 1 | 39 |
| O75462 | CRLF1 | 1 | 0 | 1 | 0 | 0 | 0 | 1 | 39 |
| P01137 | TGFB1 | 1 | 0 | 1 | 0 | 0 | 0 | 1 | 39 |
| P04040 | CAT | 1 | 0 | 1 | 0 | 0 | 0 | 1 | 39 |
| P07339 | CTSD | 1 | 0 | 1 | 0 | 0 | 0 | 1 | 39 |
| P09622 | DLD | 1 | 0 | 1 | 0 | 0 | 0 | 1 | 39 |
| O43491 | EPB41L2 | 1 | 0 | 1 | 0 | 0 | 0 | 1 | 39 |
| P28065 | PSMB9 | 1 | 0 | 1 | 0 | 0 | 0 | 1 | 39 |
| P62263 | RPS14 | 1 | 0 | 1 | 0 | 0 | 0 | 1 | 39 |
| P62424 | RPL7A | 1 | 0 | 1 | 0 | 0 | 0 | 1 | 39 |
| Q5KU26 | COLEC12 | 1 | 0 | 1 | 0 | 0 | 0 | 1 | 39 |
| Q8TAQ2 | SMARCC2 | 1 | 0 | 1 | 0 | 0 | 0 | 1 | 39 |
| P37802 | TAGLN2 | 0 | 1 | 1 | 0 | 0 | 0 | 1 | 39 |
| P50991 | CCT4 | 0 | 1 | 1 | 0 | 0 | 0 | 1 | 39 |
| Q12797 | ASPH | 0 | 1 | 1 | 0 | 0 | 0 | 1 | 39 |
| Q13098 | GPS1 | 0 | 1 | 1 | 0 | 0 | 0 | 1 | 39 |
| Q14004 | CDK13 | 0 | 1 | 1 | 0 | 0 | 0 | 1 | 39 |
| Q99962 | SH3GL2 | 0 | 1 | 1 | 0 | 0 | 0 | 1 | 39 |
| P69905 | HBA1 | 1 | 1 | 2 | 0 | 0 | 1 | 1 | 39 |
| P27695 | APEX1 | 1 | 2 | 1 | 1 | 0 | 0 | 1 | 39 |
| P28074 | PSMB5 | 1 | 2 | 0 | 0 | 0 | 0 | 1 | 39 |
| P10599 | TXN | 1 | 0 | 2 | 0 | 0 | 0 | 1 | 39 |
| P30530 | AXL | 1 | 0 | 2 | 0 | 0 | 0 | 1 | 39 |
| P62241 | RPS8 | 1 | 0 | 2 | 0 | 0 | 0 | 1 | 39 |
| Q01105 | SET | 1 | 0 | 2 | 0 | 0 | 0 | 1 | 39 |
| P62269 | RPS18 | 2 | 1 | 1 | 0 | 0 | 1 | 1 | 39 |
| P23528 | CFL1 | 2 | 1 | 2 | 1 | 0 | 1 | 1 | 39 |
| Q6E0U4 | DMKN | 2 | 1 | 0 | 0 | 0 | 0 | 1 | 39 |
| P35268 | RPL22 | 2 | 0 | 1 | 0 | 0 | 0 | 1 | 39 |
| Q02878 | RPL6 | 2 | 0 | 1 | 0 | 0 | 0 | 1 | 39 |
| Q96AY3 | FKBP10 | 2 | 0 | 1 | 0 | 0 | 0 | 1 | 39 |
| P43034 | PAFAH1B1 | 0 | 1 | 2 | 0 | 0 | 0 | 1 | 39 |
| Q99584 | S100A13 | 0 | 1 | 2 | 0 | 0 | 0 | 1 | 39 |
| P60900 | PSMA6 | 0 | 2 | 1 | 0 | 0 | 0 | 1 | 39 |
| P05121 | SERPINE1 | 1 | 1 | 3 | 0 | 0 | 0 | 1 | 39 |
| Q14444 | CAPRIN1 | 1 | 1 | 3 | 0 | 0 | 0 | 1 | 39 |
| Q16181 | SEPT7 | 1 | 3 | 1 | 1 | 0 | 0 | 1 | 39 |
| P04004 | VTN | 1 | 0 | 3 | 0 | 0 | 0 | 1 | 39 |
| Q13822 | ENPP2 | 1 | 0 | 3 | 0 | 0 | 0 | 1 | 39 |
| Q92859 | NEO1 | 1 | 0 | 3 | 0 | 0 | 0 | 1 | 39 |
| O60832 | DKC1 | 3 | 1 | 0 | 0 | 0 | 0 | 1 | 39 |
| P36222 | CHI3L1 | 3 | 1 | 0 | 0 | 0 | 0 | 1 | 39 |
| P84550 | SKOR1 | 3 | 1 | 0 | 0 | 0 | 0 | 1 | 39 |
| Q08945 | SSRP1 | 3 | 1 | 0 | 0 | 0 | 0 | 1 | 39 |
| P08572 | COL4A2 | 1 | 1 | 4 | 0 | 0 | 0 | 1 | 39 |
| P15531 | NME1 | 2 | 2 | 4 | 1 | 0 | 1 | 1 | 39 |
| P00751 | CFB | 1 | 0 | 4 | 0 | 0 | 0 | 1 | 39 |
| P41091 | EIF253 | 0 | 1 | 4 | 0 | 0 | 0 | 1 | 39 |
| Q16555 | DPYSL2 | 1 | 1 | 5 | 0 | 0 | 0 | 1 | 39 |
| Q14980 | NUMA1 | 1 | 0 | 5 | 0 | 0 | 0 | 1 | 39 |
| Q86UP2 | KTN1 | 0 | 1 | 5 | 0 | 0 | 0 | 1 | 39 |
| Q9BW59 | CHID1 | 1 | 0 | 7 | 0 | 0 | 0 | 1 | 39 |
| Q12805 | EFEMP1 | 1 | 0 | 8 | 0 | 0 | 0 | 1 | 39 |
| P63104 | YWHAZ | 1 | 1 | 9 | 0 | 0 | 0 | 1 | 39 |
| P07195 | LDHB | 0 | 1 | 10 | 0 | 0 | 0 | 1 | 39 |
| O00560 | SDCBP | 2 | 1 | 12 | 1 | 1 | 0 | 1 | 39 |
| Q9Y490 | TLN1 | 0 | 1 | 18 | 0 | 0 | 0 | 1 | 39 |
| P17661 | DES | 35 | 4 | 34 | 37 | 40 | 4 | Overlap | 40 |
| P05067 | APP | 13 | 5 | 11 | 7 | 6 | 3 | Overlap | 40 |
| Q14118 | DAG1 | 6 | 2 | 1 | 6 | 4 | 2 | Overlap | 40 |

TABLE 2
List of 25 proteins of which peptides were identified by mass spectrometry to be
consistently elevated in exosomes of ALS muscle cells compared to healthy controls, or that had
consistently high levels in both ALS and Healthy muscle exosomes, and had other features of
mechanistic relevance to the pathology. The first column gives standard protein IDs used by Uniprot
(http://www.uniprot.org/), and corresponding gene names in column 2. Columns 3-5 and 6-8 show
levels of peptides in ALS samples and healthy control samples, respectively. Column 9 indicates the
difference in median quantities of the protein peptides in ALS compared to control.

| | | | | | | | | Med |
|---|---|---|---|---|---|---|---|---|
| | | | | | | | | ALS-Cont |
| | | | | | | | | IF no |
| Uniprot | Gene | SD990- | SD1016- | GO992- | SD1048- | SD1049- | GO849- | overlap |
| ID | name | ALS1 | ALS2 | ALS3 | Cont1 | Cont2 | Cont3 | in range |
| P30101 | PDIA3 | 20 | 15 | 35 | 5 | 2 | 3 | 17 |
| Q76M96 | CCDC80 | 19 | 3 | 57 | 2 | 2 | 0 | 17 |
| P11142 | HSPA8 | 8 | 11 | 21 | 0 | 0 | 0 | 11 |
| P07093 | SERPINE2 | 11 | 5 | 16 | 1 | 3 | 0 | 10 |
| Q15149 | PLEC | 25 | 24 | 52 | 15 | 23 | 3 | 10 |
| P11021 | HSPA5 | 11 | 9 | 27 | 2 | 4 | 1 | 9 |
| P19338 | NCL | 9 | 2 | 9 | 1 | 0 | 0 | 9 |
| P09493 | TPM1 | 8 | 0 | 11 | 0 | 0 | 0 | 8 |
| P24821 | TNC | 9 | 2 | 35 | 1 | 1 | 0 | 8 |
| P17936 | IGFBP3 | 9 | 3 | 8 | 3 | 1 | 2 | 6 |
| P20908 | COL5A1 | 34 | 11 | 12 | 9 | 4 | 8 | 4 |
| Q9Y240 | CLEC11A | 8 | 5 | 7 | 3 | 4 | 1 | 4 |
| P46821 | MAP1B | 4 | 0 | 6 | 0 | 0 | 0 | 4 |
| Q13765 | NACA | 6 | 1 | 4 | 0 | 1 | 0 | 4 |
| P25787 | PSMA2 | 4 | 2 | 7 | 1 | 0 | 0 | 4 |
| P46777 | RPL5 | 4 | 3 | 4 | 1 | 1 | 1 | 3 |
| P60660 | MYL6 | 3 | 3 | 2 | 1 | 0 | 0 | 3 |
| P07858 | CTSB | 4 | 0 | 3 | 0 | 0 | 0 | 3 |
| P10809 | HSPD1 | 5 | 0 | 3 | 0 | 0 | 0 | 3 |
| Q6NZI2 | PTRF | 3 | 0 | 9 | 0 | 0 | 0 | 3 |
| Q02818 | NUCB1 | 4 | 1 | 12 | 0 | 1 | 1 | 3 |
| P13667 | PDIA4 | 3 | 3 | 16 | 1 | 0 | 0 | 3 |
| P07237 | P4HB | 3 | 2 | 29 | 0 | 0 | 0 | 3 |
| O43707 | ACTN4 | 0 | 3 | 62 | 0 | 0 | 0 | 3 |
| Q14444 | CAPRIN1 | 1 | 1 | 3 | 0 | 0 | 0 | 1 |
| P17661 | DES | 35 | 4 | 34 | 37 | 40 | 4 | Overlap |
| P05067 | APP | 13 | 5 | 11 | 7 | 6 | 3 | Overlap |
| Q14118 | DAG1 | 6 | 2 | 1 | 6 | 4 | 2 | Overlap |

### Non-Coding RNA Biomarkers

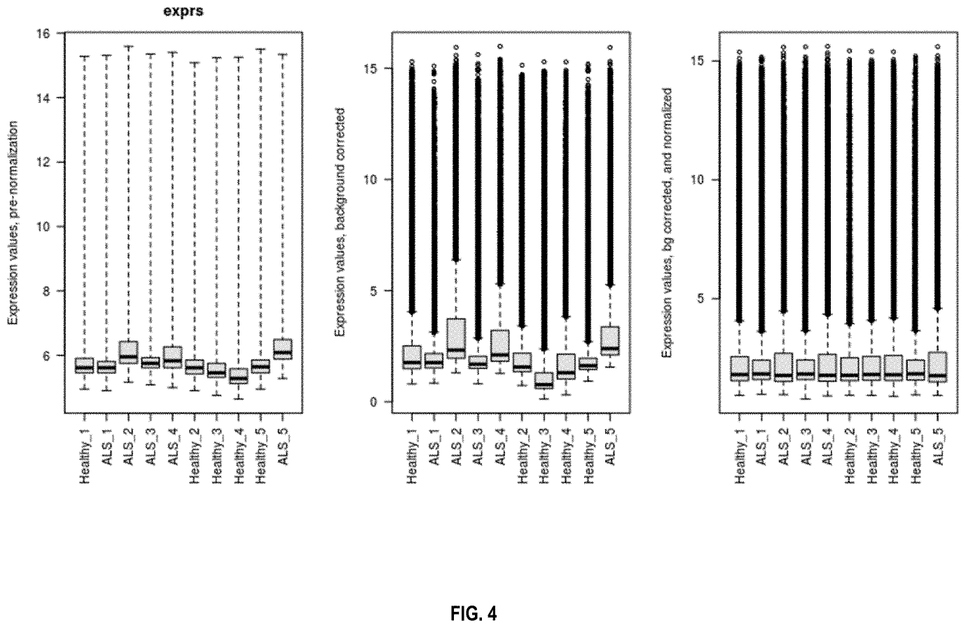

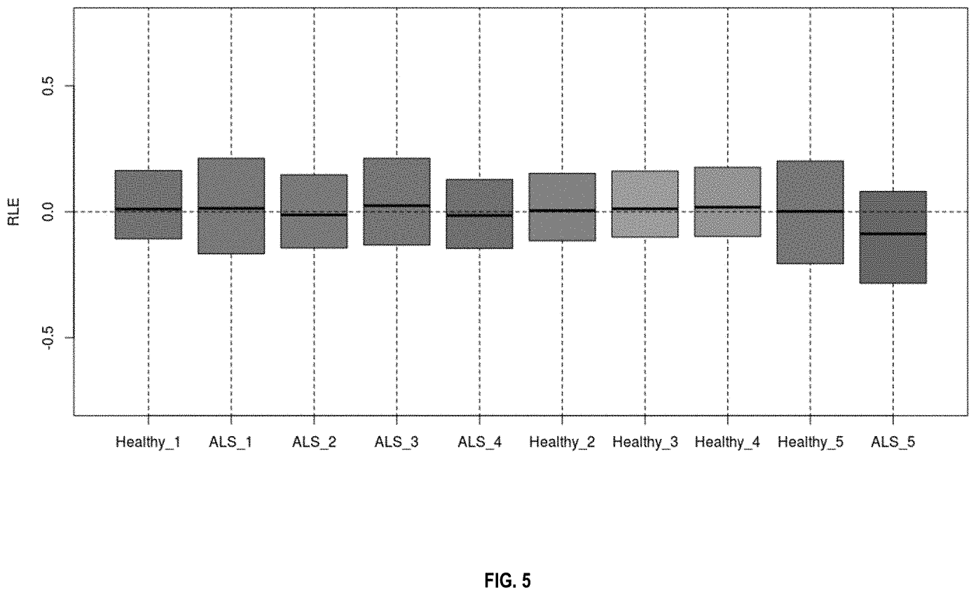

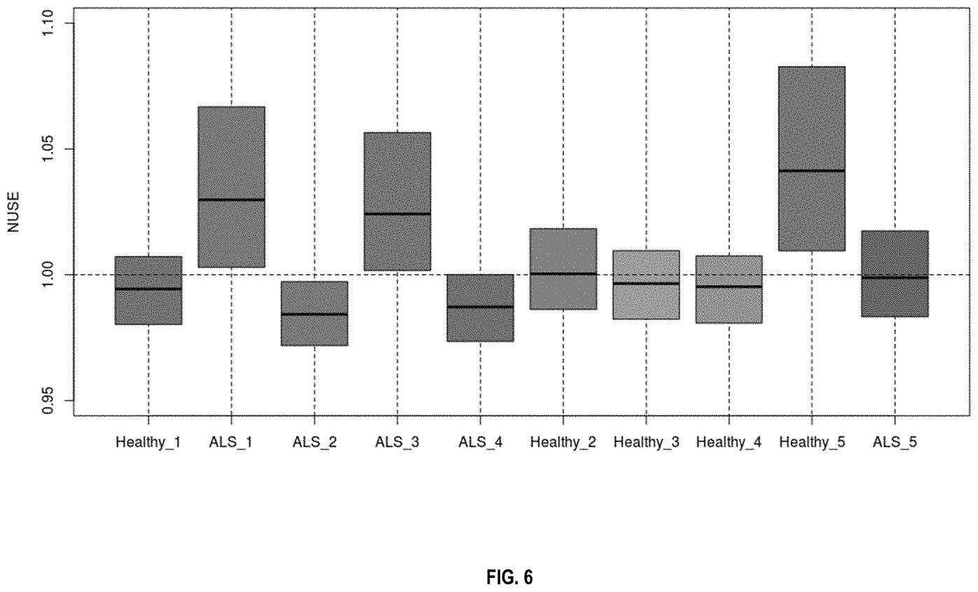

Profiling of non-coding RNA species was carried out by microarray (using Affymetrix GeneChip® miRNA 4.0 Array) on ALS exosomes (n=5) compared to healthy controls (n=5). After quality control and normalization (see FIG. 4-6), one-way unpaired ANOVA identified 14 non-coding RNAs to be significantly dysregulated in ALS v healthy controls (Table 3: 12 up-regulated in ALS, 2 down-regulated in ALS). Of these, 13 are microRNAs (indicated by their miRBase IDs hsa-*** and accession numbers MIMAT***), and 1 is the small nucleolar RNA snoU13 (ENSG00000239126).

TABLE 3
List of 14 non-coding RNAs identified to be significantly
dysregulated in exosomes of ALS muscle cells compared to healthy controls.
Fold-changes and ANOVA p-values are indicated in the right-most columns.

| | | | ALS | Healthy | | |
|---|---|---|---|---|---|---|
| | | | Bi- | Bi- | Fold | |
| | | | weight | weight | Change | ANOVA |
| | | | Avg | Avg | (linear | p-value |
| Transcript | Transcript ID | | Signal | Signal | (ALS vs. | (ALS vs. |
| Cluster ID | (Array Design) | Accession | (log2) | log(2) | Healthy) | healthy) |
| 20500422 | hsa-miR-30c-5p | MIMAT0000244 | 10.46 | 9.04 | 2.68 | 0.003772 |
| 20533735 | ENSG00000239126 | ENSG000002391 | 261.89 | 3.06 | −2.24 | 0.005702 |
| 20517693 | hsa-miR-4313 | MIMAT0016865 | 3.45 | 2.09 | 2.57 | 0.006739 |
| 20503798 | hsa-miR-493-5p | MIMAT0002813 | 4.99 | 2.28 | 6.5 | 0.013502 |
| 20501286 | hsa-miR-151a-5p | MIMAT0004697 | 10.96 | 9.9 | 2.08 | 0.01673 |
| 20500424 | hsa-miR-30d-5p | MIMAT0000245 | 8.64 | 7.13 | 2.85 | 0.023067 |
| 20501250 | hsa-miR-382-5p | MIMAT0000737 | 10.09 | 8.9 | 2.28 | 0.023902 |
| 20500163 | hsa-miR-30a-3p | MIMAT0000088 | 7.93 | 6.79 | 2.2 | 0.028053 |
| 20500713 | hsa-let-7g-5p | MIMAT0000414 | 8.91 | 7.45 | 2.74 | 0.028442 |
| 20505799 | hsa-miR-374b-5p | MIMAT0004955 | 2.87 | 1.7 | 2.25 | 0.030748 |
| 20500141 | hsa-miR-21-5p | MIMAT0000076 | 9.31 | 7.8 | 92.68 | 0.033918 |
| 20500123 | hsa-let-7f-5p | MIMAT0000067 | 6.99 | 4.94 | 4.13 | 0.040776 |
| 20517915 | hsa-miR-3663-5p | MIMAT0018084 | 2 | 3.06 | −2.08 | 0.044248 |
| 20504419 | hsa-miR-411-3p | MIMAT0004813 | 3.68 | 2.1 | 2.97 | 0.046014 |

### Example 6—Comparison of Different Biomarkers Against Different Disease Groups

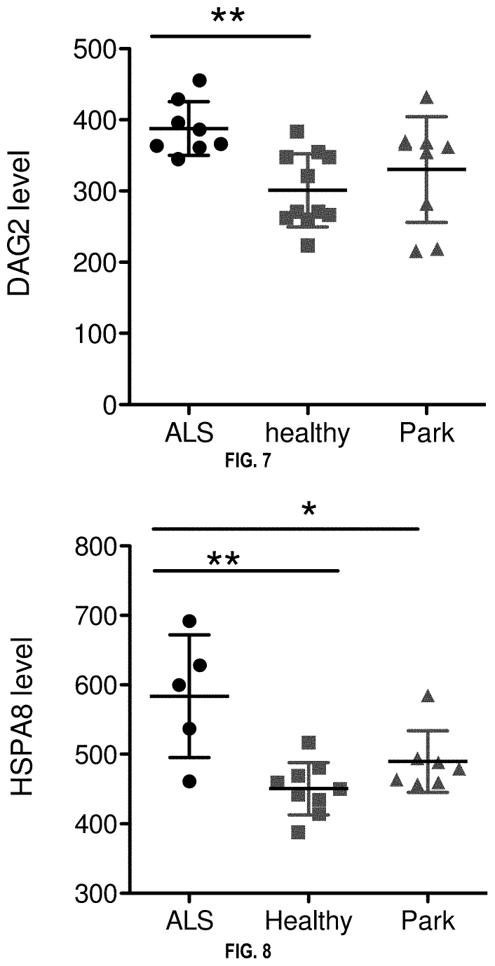

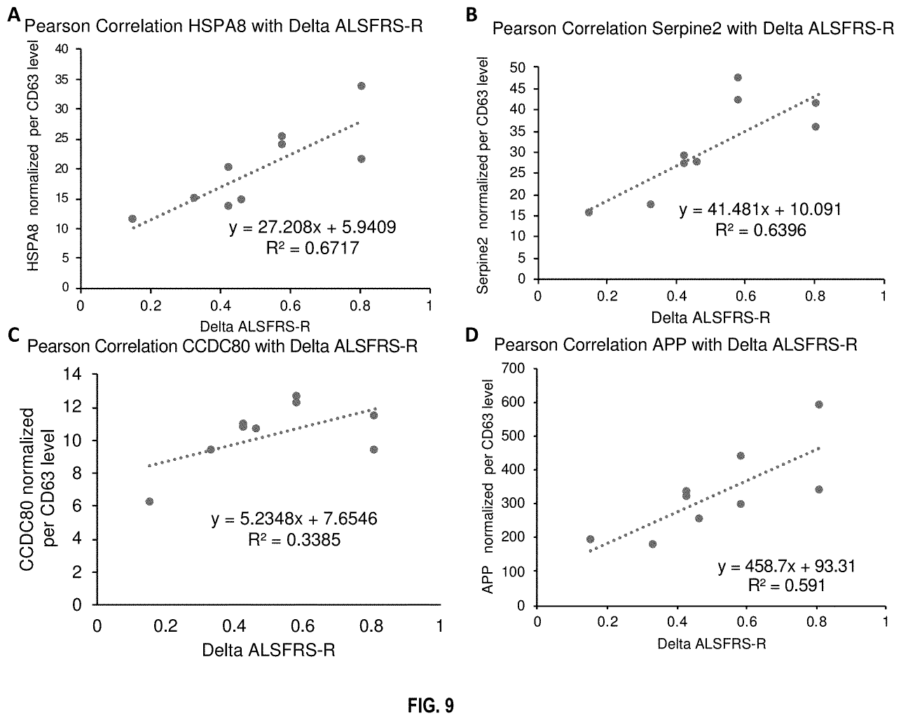

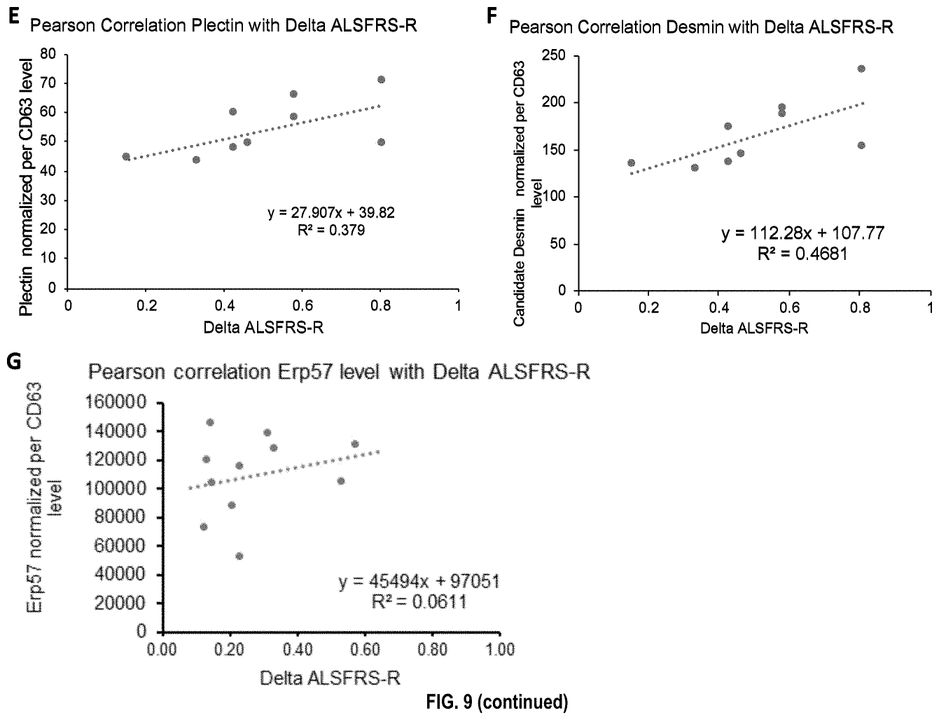

Blood samples were collected from 8 ALS patients, 11 healthy subjects and 9 Parkinson patients. The patient characteristics of the selected patients are presented in Table 4. Exosomes were extracted from serum and used for dot-blot analysis. Levels of DAG1 in exosomes isolated from the blood serum of patients with ALS disease, Parkinson's disease (disease control), or from healthy subjects are presented in FIG. 7, and levels of HSPA8 in exosomes isolated from the blood serum of patients with ALS disease, Parkinson's disease (disease control), or from healthy subjects are presented in FIG. 8. Levels of each biomarker in exosomes isolated from the blood serum of ALS patients, normalized against the quantity of total blood serum exosome (CD63) were plotted against the rate of progression of the pathology (delta ALSFRS-R) and are presented in FIG. 9.

TABLE 4
List of patient characteristics.

| | V1/ | | Bulbar: | Disease | | | |
|---|---|---|---|---|---|---|---|
| ALS/ | V6/ | | 1 | Duration | | | |
| Park/ | V12/ | ALSFRS- | Spinal: | from | Delta | | |
| Healthy | V18 | R | 2 | sign | ALSFRS | Gender | Age |
| ALS | 1 | 43 | 1 | 15 | 0.31 | M | 33 |
| ALS | 1 | 45 | 1 | 8 | 0.33 | M | 41 |
| ALS | 1 | 45 | 2 | 23 | 0.13 | M | 52 |
| ALS | 1 | 46 | 2 | 14 | 0.13 | M | 53 |
| ALS | 1 | 45 | 2 | 36 | 0.08 | M | 64 |
| ALS | 1 | 45 | 2 | 7 | 0.43 | F | 55 |
| ALS | 1 | 45 | 2 | 7 | 0.43 | M | 39 |
| ALS | 1 | 46 | 2 | 13 | 0.15 | M | 34 |
| ALS | 6 | 45 | 1 | 15 | 0.14 | M | 33 |
| ALS | 6 | 40 | 1 | 8 | 0.57 | M | 41 |
| ALS | 6 | 42 | 2 | 23 | 0.21 | M | 52 |
| ALS | 6 | 42 | 2 | 23 | 0.21 | M | 52 |
| ALS | 6 | 43 | 2 | 36 | 0.12 | M | 64 |
| ALS | 6 | 43 | 1 | 10 | 0.31 | M | 47 |
| ALS | 6 | 41 | 2 | 12 | 0.58 | F | 55 |
| ALS | 6 | 41 | 2 | 12 | 0.58 | M | 39 |
| ALS | 6 | 42 | 2 | 18 | 0.33 | M | 34 |
| ALS | 12 | 44 | 1 | 15 | 0.15 | M | 33 |
| ALS | 12 | 35 | 1 | 8 | 0.65 | M | 41 |
| ALS | 12 | 40 | 2 | 23 | 0.23 | M | 52 |
| ALS | 12 | 42 | 2 | 14 | 0.23 | M | 53 |
| ALS | 12 | 41 | 2 | 36 | 0.15 | M | 64 |
| ALS | 12 | 41 | 1 | 10 | 0.32 | M | 47 |
| ALS | 18 | 41 | 1 | 15 | 0.21 | M | 33 |
| ALS | 18 | 35 | 1 | 8 | 0.50 | M | 41 |
| ALS | 18 | 31 | 2 | 14 | 0.53 | M | 53 |
| ALS | 18 | 37 | 1 | 10 | 0.39 | M | 47 |
| ALS | 18 | 31 | 2 | 21 | 0.81 | F | 55 |
| ALS | 18 | 31 | 2 | 21 | 0.81 | M | 39 |
| ALS | 18 | 34 | 2 | 30 | 0.47 | M | 34 |
| Healthy | | | | | | F | 46 |
| Healthy | | | | | | F | 70 |
| Healthy | | | | | | F | 64 |
| Healthy | | | | | | F | 49 |
| Healthy | | | | | | F | 66 |
| Healthy | | | | | | F | 65 |
| Healthy | | | | | | F | 60 |
| Healthy | | | | | | F | 44 |
| Heathy | | | | | | F | 67 |
| Healthy | | | | | | F | 76 |
| Healthy | | | | | | F | 74 |
| Park | | | | | | F | 60 |
| Park | | | | | | M | 71 |
| Park | | | | | | F | 75 |
| Park | | | | | | F | 58 |
| Park | | | | | | F | 71 |
| Park | | | | | | M | 45 |
| Park | | | | | | F | 66 |
| Park | | | | | | M | 66 |
| Park | | | | | | F | 63 |

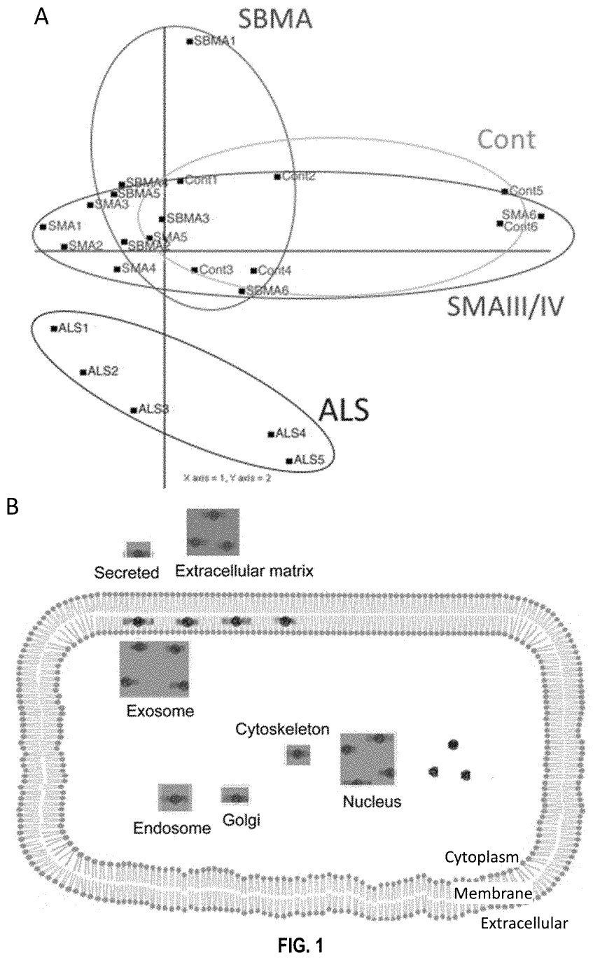

The present invention provides a robust ALS gene expression signature and a new driver of toxicity in muscle cells and muscle-motor neuron interactions in ALS patients. By studying differentiated muscle (myotube) cells from muscle biopsies of sporadic ALS patients, the present invention shows: (1) the gene expression profiles of myotubes isolated from ALS patients cluster entirely separately not only from healthy control subjects but also from SBMA and SMA-III/IV disease controls (see FIG. 1A). The 30 genes most strongly contributing to this ALS-specific signature include genes encoding for proteins secreted or localized in exosomes (see FIG. 1B); (2) both intracellular exosomal biogenesis and extracellular exosomal secretion are dramatically increased in ALS myotubes (see FIGS. 2A and B), and exosome-rich multi-vesicular bodies are observed by electron microscopy in muscle biopsies of ALS patients (see FIG. 2C); (3) increased exosomal biogenesis and secretion are not observed in myotubes of muscle-denervated human subjects (neither healthy, SBMA, nor SMA-III/IV) or of muscle-denervated healthy murine muscle (see FIG. 1, 2A and other data not shown), indicating that exosomal secretion is not a simple downstream consequence of the disease.

1. A method of diagnosing or prognosing a neurological disorder in a subject, the method comprising the steps of:
(a) determining the quantitative or qualitative level of one or more biomarkers in a biological sample from the subject; and
(b) diagnosing or prognosing the neurological disorder in the subject based on the quantitative or qualitative level of the or each biomarker in the biological sample;
wherein the or each biomarker is selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; CAPRIN1; DES; APP; and DAG1.

2. The method according to claim 1, wherein the determining step (a) comprises determining the quantitative or qualitative level of all of the biomarkers in the biological sample from the subject.

3. The method according to claim 1, wherein the determining step (a) comprises determining the quantitative or qualitative level of each of the biomarkers in the biological sample from the subject.

4. The method according to claim 1,
wherein the or each biomarker is a gene.

5. The method according to claim 4, wherein the or each biomarker is a gene selected from: PDIA3; CCDC80; HSPA8; SERPINE2; PLEC; DES; APP; and DAG1.

6. The method according to claim 1, wherein the or each biomarker is a protein encoded by a gene selected from: PDIA3; CCDC80; HSPA8; SERPINE2; PLEC; HSPA5; NCL; TPM1; TNC; IGFBP3; COL5A1; CLEC11A; MAP1B; NACA; PSMA2; RPL5; MYL6; CTSB; HSPD1; PTRF; NUCB1; PDIA4; P4HB; ACTN4; CAPRIN1; DES; APP; and DAG1.

7. The method according to claim 6, wherein the or each biomarker is a protein encoded by a gene selected from: PDIA3; CCDC80; HSPA8; SERPINE2; PLEC; and DAG1

8. The method according to claim 6, wherein the or each biomarker is a protein having a UniProtKB/Swiss-Prot Accession Number selected from: P30101; Q76M96; P11142; P07093; Q15149; P11021; P19338; P09493; P24821; P17936; P20908; Q9Y240; P46821; Q13765; P25787; P46777; P60660; P07858; P10809; Q6NZI2; Q02818; P13667; P07237; 043707; Q14444; P17661; P05067; and Q14118.

9. The method according to claim 8, wherein the or each biomarker is a protein having a UniProtKB/Swiss-Prot Accession Number selected from: P30101; Q76M96; P11142; P07093; Q15149; P17661; P05067; and Q14118.

10. The method according to claim 1, wherein the neurological disorder is a neurodegenerative disorder.

11. The method according to claim 10, wherein the neurodegenerative disorder is selected from amyotrophic lateral sclerosis (ALS; Lou Gehrig's disease; motor neurone disease; MND), hereditary spastic paraplegia (HSP), primary lateral sclerosis (PLS), progressive muscular atrophy (PMA), progressive bulbar palsy (PBP), pseudobulbar palsy, spinal-bulbar muscular atrophy (SBMA), and spinal muscular atrophy.

12. The method according to claim 1, wherein the diagnosing or prognosing step (b) comprises comparing the quantitative or qualitative level of the or each biomarker in the biological sample from the subject with the quantitative or qualitative level of the or each respective biomarker in a normal sample.

13. The method according to claim 12, wherein a quantitative or qualitative level of the or each biomarker in the biological sample from the subject greater than the quantitative or qualitative level of the or each respective biomarker in a normal sample is indicative of the quantitative or qualitative presence of the neurological disorder.

14. The method according to claim 1, wherein the biological sample is selected from whole blood, serum, plasma, urine, interstitial fluid, peritoneal fluid, cervical swab, tears, saliva, buccal swab, skin, brain tissue, and cerebrospinal fluid.

15. The method according to claim 1, wherein the biological sample is whole blood.
